(19) United States
(12) Patent Application Publication
(10) Pub. No.: US 2008/0319276 A1
(43) Pub. Date: Dec. 25, 2008

(54) Computational user-health testing

(75) Inventors: Edward K.Y. Jung, Bellevue, WA (US); Eric C. Leuthardt, St. Louis, MO (US); Royce A. Levien, Lexington, MA (US); Robert W. Lord, Seattle, WA (US); Mark A. Malamud, Seattle, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/156,663

(22) Filed: Jun. 2, 2008

Related U.S. Application Data
(63) Continuation-in-part of application No. 11/807,220, filed on May 24, 2007.
Application No. 11/807,220 is a continuation-in-part of application No. 11/731,801, filed on Mar. 30, 2007.
Application No. 11/731,801 is a continuation-in-part of application No. 11/731,778, filed on Mar. 30, 2007.
Application No. 11/731,778 is a continuation-in-part of application No. 11/731,745, filed on Mar. 30, 2007.

(51) Int. Cl. A61B 5/00; G06Q 50/00

(52) U.S. Cl. 600/300; 705/3

(57) ABSTRACT

Methods, apparatuses, computer program products, devices and systems are described that carry out detecting user data from an interaction between a user and at least one device-implemented application whose primary function is different from symptom detection; mapping the user data from the interaction between the user and the at least one device-implemented application whose primary function is different from symptom detection to at least one user-health test function set; and selecting at least one user-health test function in response to the at least one user-health test function set.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

Related Applications:
For purposes of the USPTO extra-statutory requirements, the present application constitutes a continuation-in-part of U.S. patent application Ser. No. NOT YET ASSIGNED, entitled COMPUTATIONAL USER-HEALTH TESTING, naming Edward K. Y. Jung; Eric C. Leuthardt; Royce A. Levien; Robert W. Lord; and Mark A. Malamud as inventors, filed 15 May 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,745, entitled EFFECTIVE RESPONSE PROTOCOLS FOR HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,778, entitled CONFIGURING SOFTWARE FOR EFFECTIVE HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,801, entitled EFFECTIVE LOW PROFILE HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

This description relates to data capture and data handling techniques.

### SUMMARY

An embodiment provides a method. In one implementation, the method includes but is not limited to detecting user data from an interaction between a user and at least one device-implemented application whose primary function is different from symptom detection; mapping the user data from the interaction between the user and the at least one device-implemented application whose primary function is different from symptom detection to at least one user-health test function set; and selecting at least one user-health test function in response to the at least one user-health test function set. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

An embodiment provides a computer program product. In one implementation, the computer program product includes but is not limited to a signal-bearing medium bearing (a) one or more instructions for detecting user data from an interaction between a user and at least one device-implemented application whose primary function is different from symptom detection; (b) one or more instructions for mapping the user data from the interaction between the user and the at least one device-implemented application whose primary function is different from symptom detection to at least one user-health test function set; and (c) one or more instructions for selecting at least one user-health test function in response to the at least one user-health test function set. In addition to the foregoing, other computer program product aspects are described in the claims, drawings, and text forming a part of the present disclosure.

An embodiment provides a system. In one implementation, the system includes but is not limited to a computing device and instructions. The instructions when executed on the computing device cause the computing device to (a) detect user data from an interaction between a user and at least one device-implemented application whose primary function is different from symptom detection; (b) map the user data from the interaction between the user and the at least one device-implemented application whose primary function is different from symptom detection to at least one user-health test function set; and (c) select at least one user-health test function in response to the at least one user-health test function set. In addition to the foregoing, other system aspects are described in the claims, drawings, and text forming a part of the present disclosure.

In one or more various aspects, related systems include but are not limited to computing means and/or programming for effecting the herein-referenced method aspects; the computing means and/or programming may be virtually any combination of hardware, software, and/or firmware configured to effect the herein-referenced method aspects depending upon the design choices of the system designer.

In addition to the foregoing, various other method and/or system and/or program product aspects are set forth and described in the teachings such as text (e.g., claims and/or detailed description) and/or drawings of the present disclosure.

The foregoing is a summary and thus contains, by necessity, simplifications, generalizations and omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE DRAWINGS

With reference now to FIG. 1, shown is an example of a user interaction and data processing system in which embodiments may be implemented, perhaps in a device, which may serve as a context for introducing one or more processes and/or devices described herein.

FIG. 2 illustrates certain alternative embodiments of the data capture and processing system of FIG. 1.

FIG. 3 illustrates certain alternative embodiments of the data capture and processing system of FIG. 1.

With reference now to FIG. 4, shown is an example of an operational flow representing example operations related to computational user-health testing, which may serve as a context for introducing one or more processes and/or devices described herein.

FIG. 5 illustrates an alternative embodiment of the example operational flow of FIG. 4.

FIG. 6 illustrates an alternative embodiment of the example operational flow of FIG. 4.

FIG. 7 illustrates an alternative embodiment of the example operational flow of FIG. 4.

FIG. 8 illustrates an alternative embodiment of the example operational flow of FIG. 4.

FIG. 9 illustrates an alternative embodiment of the example operational flow of FIG. 4.

FIG. 10 illustrates an alternative embodiment of the example operational flow of FIG. 4.

With reference now to FIG. 11, shown is a partial view of an example computer program product that includes a computer program for executing a computer process on a computing device related to computational user-health testing, which may serve as a context for introducing one or more processes and/or devices described herein.

With reference now to FIG. 12, shown is an example device in which embodiments may be implemented related to computational user-health testing, which may serve as a context for introducing one or more processes and/or devices described herein.

The use of the same symbols in different drawings typically indicates similar or identical items.

### DETAILED DESCRIPTION

FIG. 1 illustrates an example system 100 in which embodiments may be implemented. The system 100 includes at least one device 102. The at least one device 102 may contain, for example, an application 104 and a user data mapping unit 140. Through interaction with application 104, user 190 may generate user data 116 that may be obtained by the at least one device 102 and/or user data mapping unit 140.

The user data mapping unit 140 may include one or more user-health test function sets, for example, user-health test function set 196, user-health test function set 197, and/or user-health test function set 198.

The device 102 may optionally include a data detection module 114, a data capture module 136, and/or a user-health test function selection module 138. The system 100 may also include a user input device 180, and/or a user monitoring device 182.

In some embodiments the user data mapping unit 140 and/or user-health test function selection module 138 may be located on an external device 194 that can communicate with the at least one device 102, on which the application 104 is operable, via network 192.

In FIG. 1, the at least one device 102 is illustrated as possibly being included within a system 100. Of course, virtually any kind of computing device may be used in connection with the application 104, such as, for example, a workstation, a desktop computer, a mobile computer, a networked computer, a collection of servers and/or databases, or a tablet PC.

Additionally, not all of the application 104, user data mapping unit 140, and/or user-health test function selection module 138 need be implemented on a single computing device. For example, the application 104 may be implemented and/or operable on a remote computer, while the user interface 184 and/or user data 116 are implemented and/or stored on a local computer as the at least one device 102. Further, aspects of the application 104, user data mapping unit 140 and/or user-health test function selection module 138 may be implemented in different combinations and implementations than that shown in FIG. 1. For example, functionality of the user interface 184 may be incorporated into the at least one device 102. The at least one device 102, user data mapping unit 140, and/or user-health test function selection module 138 may perform simple data relay functions and/or complex data analysis, including, for example, fuzzy logic and/or traditional logic steps. Further, many methods of searching databases known in the art may be used, including, for example, unsupervised pattern discovery methods, coincidence detection methods, and/or entity relationship modeling. In some embodiments, the at least one device 102, user data mapping unit 140, and/or user-health test function selection module 138 may process user data 116 according to health profiles available as updates through a network.

The user data 116 may be stored in virtually any type of memory that is able to store and/or provide access to information in, for example, a one-to-many, many-to-one, and/or many-to-many relationship. Such a memory may include, for example, a relational database and/or an object-oriented database, examples of which are provided in more detail herein.

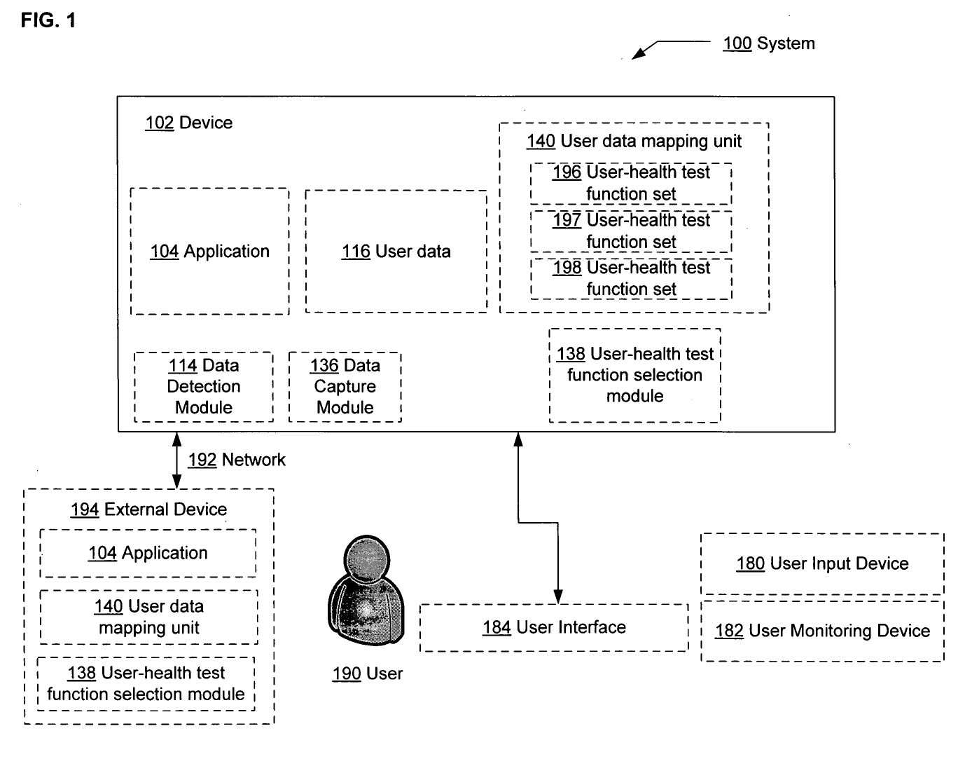

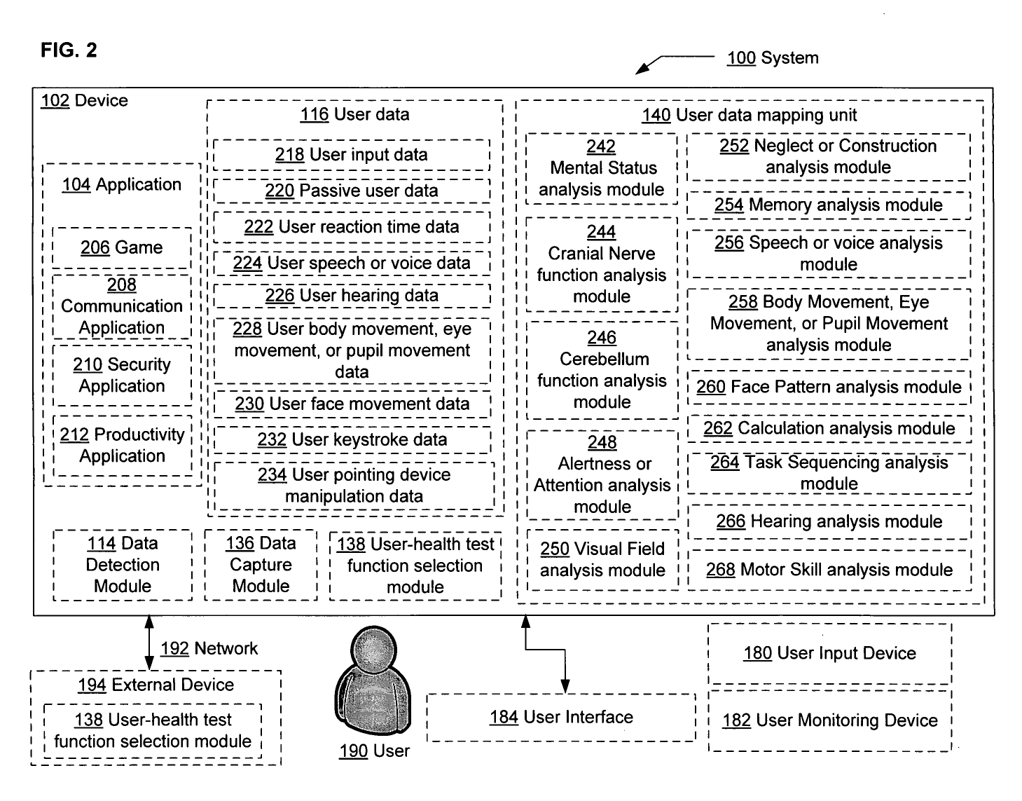

FIG. 2 illustrates an example system 100 in which embodiments may be implemented. The system 100 includes at least one device 102. The at least one device 102 may contain, for example, an application 104 and a user data mapping unit 140. Through interaction with application 104, user 190 may generate user data 116 that may be obtained by the at least one device 102 and/or user data mapping unit 140. The application 104 may include, for example, a game 206, a communication application 208, a security application 210, and/or a productivity application 212. User data 116 may include, for example, user input data 218, passive user data 220, user reaction time data 222, user speech or voice data 224, user hearing data 226, user body movement, pupil movement, or eye movement data 228, user face movement data 230, user keystroke data 232, and/or user pointing device manipulation data 234.

The user data mapping unit 140 may include, for example, mental status analysis module 242; cranial nerve function analysis module 244; cerebellum function analysis module 246; alertness or attention analysis module 248; visual field analysis module 250; neglect or construction analysis module 252; memory analysis module 254; speech or voice analysis module 256; body movement, eye movement, or pupil movement analysis module 258; face pattern analysis module 260; calculation analysis module 262; task sequencing analysis module 264; hearing analysis module 266; and/or motor skill analysis module 268. The user data 116 may be stored in virtually any type of memory that is able to store and/or provide access to information in, for example, a one-to-many, many-to-one, and/or many-to-many relationship. Such a memory may include, for example, a relational database and/or an object-oriented database, examples of which are provided in more detail herein.

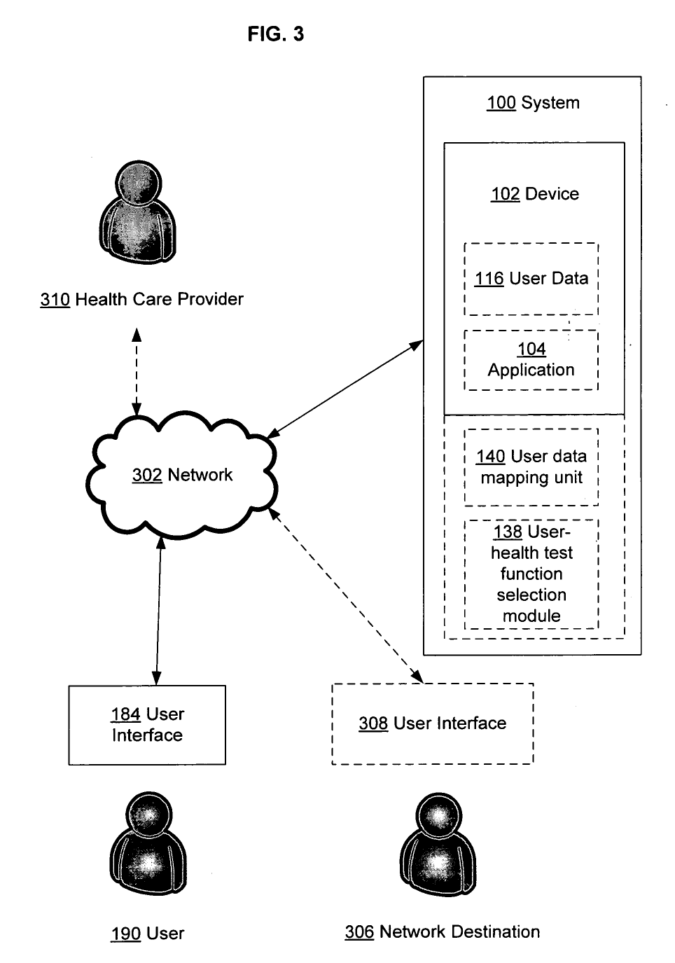

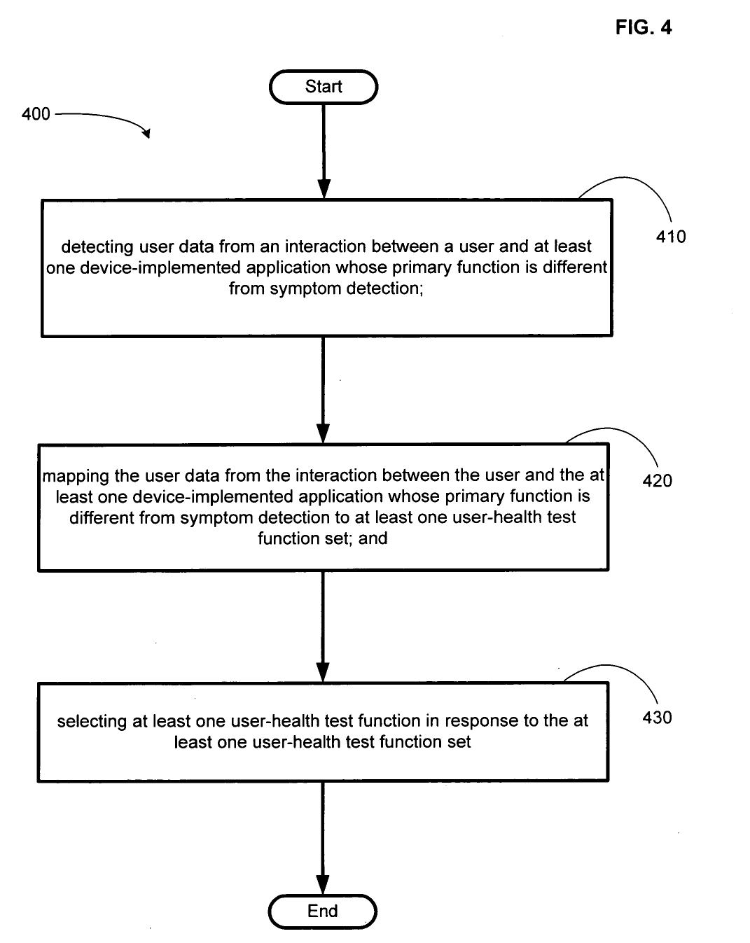

FIG. 3 illustrates certain alternative embodiments of the system 100 of FIG. 1. In FIG. 3, the user 190 may use the user interface 184 to interact through a network 302 with the application 104 operable on the at least one device 102. A user data mapping unit 140 and/or user-health test function selection module 138 may be implemented on the at least one device 102, or elsewhere within the system 100 but separate from the at least one device 102. The at least one device 102 may be in communication over a network 302 with a network destination 306 and/or healthcare provider 310, which may interact with the at least one device 102, user data mapping unit 140, and/or user-health test function selection module 138 through, for example, a user interface 308. Of course, it should be understood that there may be many users other than the specifically-illustrated user 190, for example, each with access to a local instance of the application 104.

In this way, the user 190, who may be using a device that is connected through a network 302 with the system 100 (e.g., in an office, outdoors and/or in a public environment), may generate user data 116 as if the user 190 were interacting locally with the at least one device 102 on which the application 104 is locally operable.

As referenced herein, the at least one device 102 and/or user-health test function selection module 138 may be used to perform various data querying and/or recall techniques with respect to the user data 116, in order to select at least one user-health test function in response to the at least one user-health test function set. For example, where the user data 116 is organized, keyed to, and/or otherwise accessible using one or more reference health condition attributes or profiles, various Boolean, statistical, and/or semi-boolean searching techniques may be performed to match user data 116 with reference health condition data, attributes, or profiles.

Many examples of databases and database structures may be used in connection with the at least one device 102, user data mapping unit 140, and/or user-health test function selection module 138. Such examples include hierarchical models (in which data is organized in a tree and/or parent-child node structure), network models (based on set theory, and in which multi-parent structures per child node are supported), or object/relational models (combining the relational model with the object-oriented model).

Still other examples include various types of eXtensible Mark-up Language (XML) databases. For example, a database may be included that holds data in some format other than XML, but that is associated with an XML interface for accessing the database using XML. As another example, a database may store XML data directly. Additionally, or alternatively, virtually any semi-structured database may be used, so that context may be provided to/associated with stored data elements (either encoded with the data elements, or encoded externally to the data elements), so that data storage and/or access may be facilitated.

Such databases, and/or other memory storage techniques, may be written and/or implemented using various programming or coding languages. For example, object-oriented database management systems may be written in programming languages such as, for example, C++ or Java. Relational and/or object/relational models may make use of database languages, such as, for example, the structured query language (SQL), which may be used, for example, for interactive queries for information and/or for gathering and/or compiling data from the relational database(s).

For example, SQL or SQL-like operations over one or more of reference health condition may be performed, or Boolean operations using a reference health condition may be performed. For example, weighted Boolean operations may be performed in which different weights or priorities are assigned to one or more of the reference health conditions, perhaps relative to one another. For example, a number-weighted, exclusive-OR operation may be performed to request specific weightings of desired (or undesired) health reference data to be included or excluded.

FIG. 4 illustrates an operational flow 400 representing example operations related to computational user-health testing. In FIG. 4 and in following figures that include various examples of operational flows, discussion and explanation may be provided with respect to the above-described system environments of FIGS. 1-3, and/or with respect to other examples and contexts. However, it should be understood that the operational flows may be executed in a number of other environment and contexts, and/or in modified versions of FIGS. 1-3. Also, although the various operational flows are presented in the sequence(s) illustrated, it should be understood that the various operations may be performed in other orders than those which are illustrated, or may be performed concurrently.

After a start operation, operation 410 shows detecting user data from an interaction between a user and at least one device-implemented application whose primary function is different from symptom detection. The user data 116 may be detected by a data detection module 114 resident on at least one device 102 or otherwise associated with a system 100. Alternatively, user data 116 may be detected by a user input device 180 and/or user monitoring device 182 associated with the at least one device 102 and/or system 100. Alternatively user data 116 may be detected by a data capture module 136 associated with the at least one device 102 and/or system 100.

System 100 and/or the at least one device 102 may also include application 104 that is operable on the at least one device 102, to perform a primary function that is different from symptom detection. For example, an online computer game may be operable as an application 104 on a personal computing device through a network. Thus the at least one application 104 may reside on the at least one device 102, or the at least one application 104 may not reside on the at least one device 102 but instead be operable on the at least one device 102 from a remote location, for example, through a network or other link.

User data 116 may include various types of user data, including but not limited to user input data 218, passive user data 220, user reaction time data 222, user speech or voice data 224, user hearing data 226, user body movement, pupil movement, or eye movement data 228, user face movement data 230, user keystroke data 232, and/or user pointing device manipulation data 234. For example, where a user interacts with an online computer game on a personal computing device, some or all of the following user data 116 may be detectable: user input data 218 in the form of security keys entered to begin the game, or level of difficulty selected for the game session; user reaction time data 222 in the form of mouse movement speed in reaching an on-screen target; user keystroke data 232 in the form of text entry in response to game prompts, including interactions with other characters in the online game; or mouse operation by the user in navigating a course through the game world/environment.

Operation 420 depicts mapping the user data from the interaction between the user and the at least one device-implemented application whose primary function is different from symptom detection to at least one user-health test function set. For example, a user data mapping unit 140 of the at least one device 102, or associated with the at least one device 102, may map user data 116 detected from the interaction between the user 190 and the application 104 to at least one user-health test function set 196, user-health test function set 197, and/or user-health test function set 198. For example, the user data mapping unit 140 may map user reaction time data 222 to an alertness or attention analysis module 248 containing a user-health test function set that can make use of the reaction time data 222. The alertness or attention analysis module 248 may contain a specific user-health test function set 196, including various alertness or attention test functions described below, such as a reaction time test function and/or a test of a user's ability to say a series of numbers forward and backwards.

Operation 430 depicts selecting at least one user-health test function in response to the at least one user-health test function set. For example, the at least one device 102 and/or user-health test function selection module 138 may select a particular user-health test function from a user-health test function set 196, for example, based on a match between the user data type, e.g., speech data, and the user-health test function set, e.g., a user speech test function within a speech or voice analysis module 256. Selecting at least one user-health test function in response to the at least one user-health test function set may also be carried out based on a user preference or a default setting, for example.

User data signals may first be encoded and/or represented in digital form (i.e., as digital data), prior to the assignment to at least one memory. For example, a digitally-encoded representation of user eye movement data may be stored in a local memory, or may be transmitted for storage in a remote memory.

Thus, an operation may be performed relating either to a local or remote storage of the digital data, or to another type of transmission of the digital data. Operations also may be performed relating to accessing, querying, processing, recalling, or otherwise obtaining the digital data from a memory, including, for example, receiving a transmission of the digital data from a remote memory. Accordingly, such operation(s) may involve elements including at least an operator (e.g., either human or computer) directing the operation, a transmitting computer, and/or a receiving computer, and should be understood to occur within the United States as long as at least one of these elements resides in the United States.

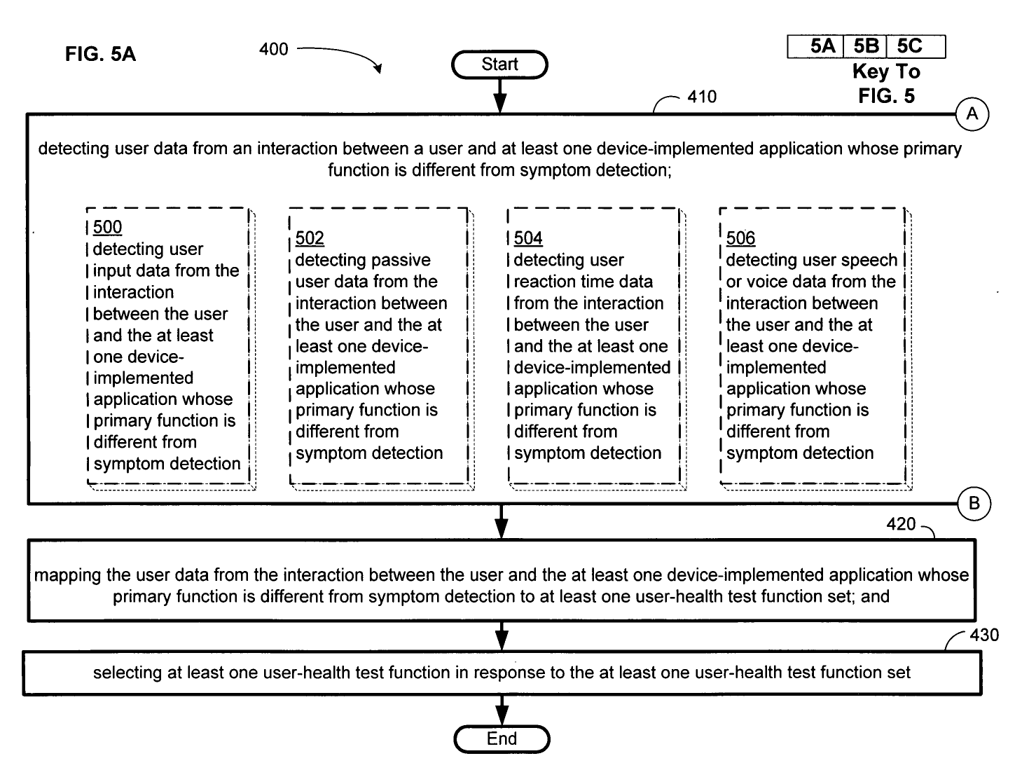

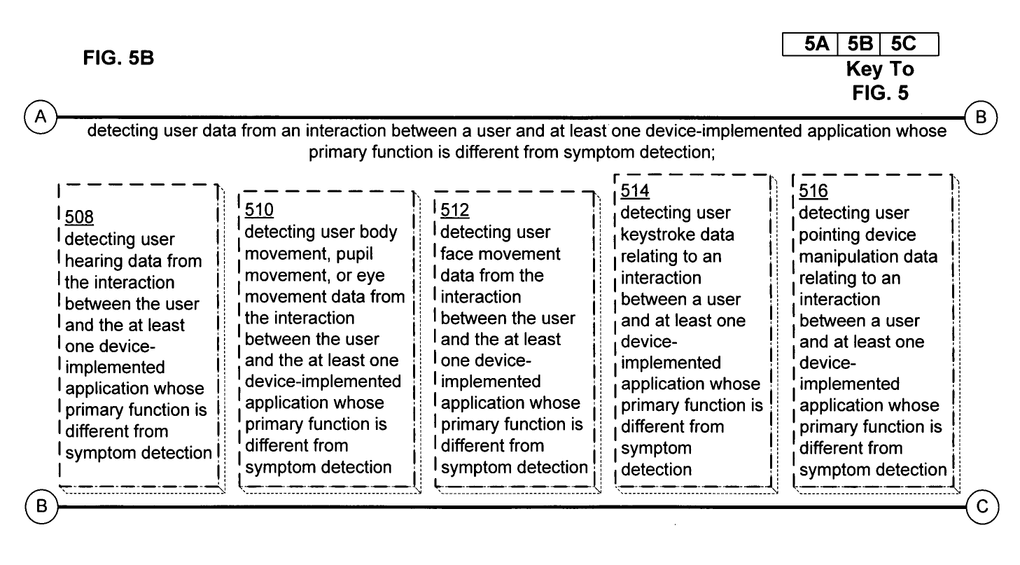

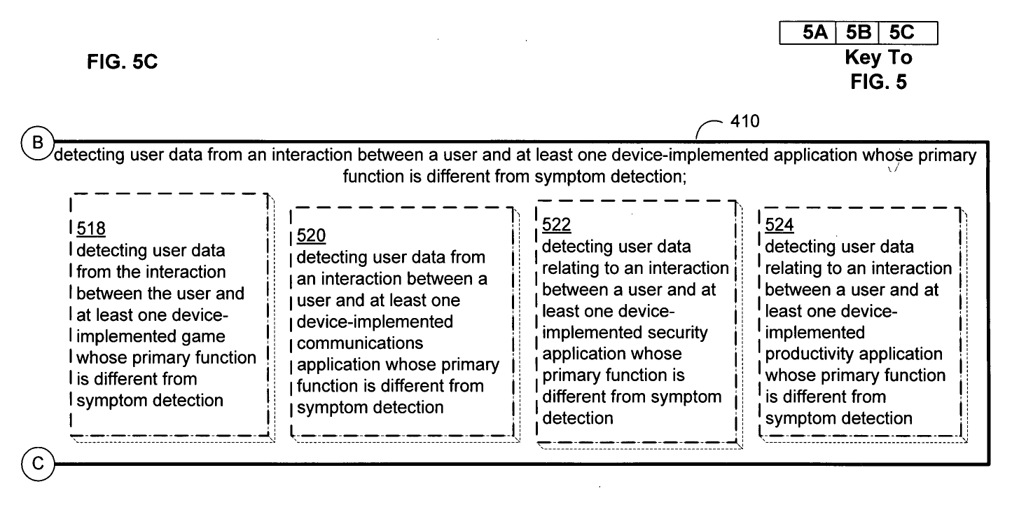

FIG. 5 illustrates alternative embodiments of the example operational flow 400 of FIG. 4. FIG. 5 illustrates example embodiments where the implementing operation 410 may include at least one additional operation. Additional operations may include operation 500, 502, 504, 506, 508, 510, 512, 514, 516, 518, 520, 522, and/or operation 524.

Operation 500 depicts detecting user input data from the interaction between the user and the at least one device-implemented application whose primary function is different from symptom detection. For example, the at least one device 102 and/or data detection module 114 may detect user input data of a certain type, for example, user speech input through a microphone user interface during an interaction between the user 190 and a speech recognition application operable on the at least one device 102.

Operation 502 depicts detecting passive user data from the interaction between the user and the at least one device-implemented application whose primary function is different from symptom detection. For example, the at least one device 102 and/or data capture module 136 may detect passive user data of a certain type, for example, user face movement data acquired by a camera set up to monitor the user during interaction with, for example, a game 206 that is operable on the at least one device 102. Another example of passive user data is flushing, blushing, or other skin color change in the user that can be detected by, for example, a camera.

Operation 504 depicts detecting user reaction time data from the interaction between the user and the at least one device-implemented application whose primary function is different from symptom detection. For example, the at least one device 102 and/or user input device 180 may detect user reaction time data from an interaction between the user and a game 206 that is operable on the at least one device 102. For example, the reaction time data may be detectable in terms of mouse movement from point A to point B on a display within a given time interval, or it may be detectable in terms of the time between a system prompt for the user to click an item on a display and the user action (e.g., moving the mouse and/or clicking the item on the display).

Operation 506 depicts detecting user speech or voice data from the interaction between the user and the at least one device-implemented application whose primary function is different from symptom detection. For example, the at least one device 102 and/or user monitoring device 182 may detect user voice data during an interaction between a user 190 and a game 206 that involves voice communication with, for example, online teammates. Alternatively, for example, the at least one device 102 and/or user monitoring device 182 may detect user voice data during an interaction between a user 190 and a telephony application operable on a mobile telephone.

Operation 508 depicts detecting user hearing data from the interaction between the user and the at least one device-implemented application whose primary function is different from symptom detection. For example, the at least one device 102 and/or user monitoring device 182 may detect user hearing data from an interaction between a user 190 and a music-playing application by measuring sound volume settings or changes thereto. Alternatively, for example, the at least one device 102 and/or user monitoring device 182 may detect user hearing data from an interaction between the user 190 and a mobile telephone by determining a volume setting on the telephone or changes to the volume setting.

Operation 510 depicts detecting user body movement, pupil movement, or eye movement data from the interaction between the user and the at least one device-implemented application whose primary function is different from symptom detection. For example, the at least one device 102 and/or user monitoring device 182 may detect user pupil movement data during a user's interaction with a videoconferencing application operable on the at least one device 102. Alternatively, for example, the at least one device 102 and/or user monitoring device 182 may detect user body movement data during an interaction between the user 190 and a game involving user motion, for example swinging a bat in a virtual baseball game.

Operation 512 depicts detecting user face movement data from the interaction between the user and the at least one device-implemented application whose primary function is different from symptom detection. For example, the at least one device 102, data capture module 136, and/or user monitoring device 182 may detect user face movement data from an interaction between the user 190 and a videoconferencing application. Another example of user face movement data is flushing, blushing, or other skin color change in the user's face that can be detected by, for example, a camera.

Operation 514 depicts detecting user keystroke data relating to an interaction between a user and at least one device-implemented application whose primary function is different from symptom detection. For example, the at least one device 102, data detection module 114, and/or user input device 180 may detect user keystroke data during an interaction between the user 190 and a word processing program, or an email program on a handheld device. Alternatively, for example, the at least one device 102, data detection module 114, and/or user input device 180 may detect user keystroke data during an interaction between the user 190 and a telephony application on a mobile telephone. User keystroke data may include typing rate, response time as detected by keystroke input, or the like.

Operation 516 depicts detecting user pointing device manipulation data relating to an interaction between a user and at least one device-implemented application whose primary function is different from symptom detection. For example, the at least one device 102, data detection module 114, and/or user input device 180 may detect user pointing device manipulation data during an interaction between the user 190 and a game 206 that involves mouse, trackball, stylus movement, or the like.

Operation 518 depicts detecting user data from the interaction between the user and at least one device-implemented game whose primary function is different from symptom detection. For example, the at least one device 102, data detection module 114, and/or user input device 180 may detect user data 116 from an interaction between the user 190 and at least one puzzle game operable on the at least one device. Such a game 206 may generate user data 116 via a user input device 180 and/or user monitoring device 182. Examples of a user input device 180 include a text entry device such as a keyboard, a pointing device such as a mouse, a touchscreen, or the like. Examples of a user monitoring device 182 include a microphone, a photography device, a video device, or the like.

Examples of a game 206 may include a computer game such as, for example, solitaire, puzzle games, role-playing games, first-person shooting games, strategy games, sports games, racing games, adventure games, or the like. Such games may be played offline or through a network (e.g., online games). Other examples of a game 206 include games involving physical gestures, and interactive games.

Operation 520 depicts detecting user data from an interaction between a user and at least one device-implemented communications application whose primary function is different from symptom detection. For example, the at least one device 102, data detection module 114, and/or user input device 180 may detect user data 116 from an interaction between the user 190 and at least one communication application 208. Such a communication application 208 may generate user data 116 via a user input device 180 and/or a user monitoring device 182.

Examples of a communication application 208 may include various forms of one-way or two-way information transfer, typically to, from, between, or among devices. Some examples of communications applications include: an email program, a telephony application, a video communications function, an internet or other network messaging program, a cell phone communication application, or the like. Such a communication application may operate via text, voice, video, or other means of communication, combinations of these, or other means of communication.

Operation 522 depicts detecting user data relating to an interaction between a user and at least one device-implemented security application whose primary function is different from symptom detection. For example, the at least one device 102, data detection module 114, user monitoring device 182, and/or user input device 180 may detect user data 116 from an interaction between the user 190 and at least one security application 210. Such a security application 210 may generate user data 116 via a user input device 146 or a user monitoring device 148.

Examples of a security application 210 may include a password entry program, a code entry system, a biometric identification application, a video monitoring system, or the like.

Operation 524 depicts detecting user data relating to an interaction between a user and at least one device-implemented productivity application whose primary function is different from symptom detection. For example, the at least one device 102, data detection module 114, and/or user input device 180 may detect user data 116 from an interaction between the user 190 and at least one productivity application 212. Such a productivity application 212 may generate user data 116 via a user input device 180 and/or a user monitoring device 182.

Examples of a productivity application 212 may include a word processing program, a spreadsheet program, business software, or the like.

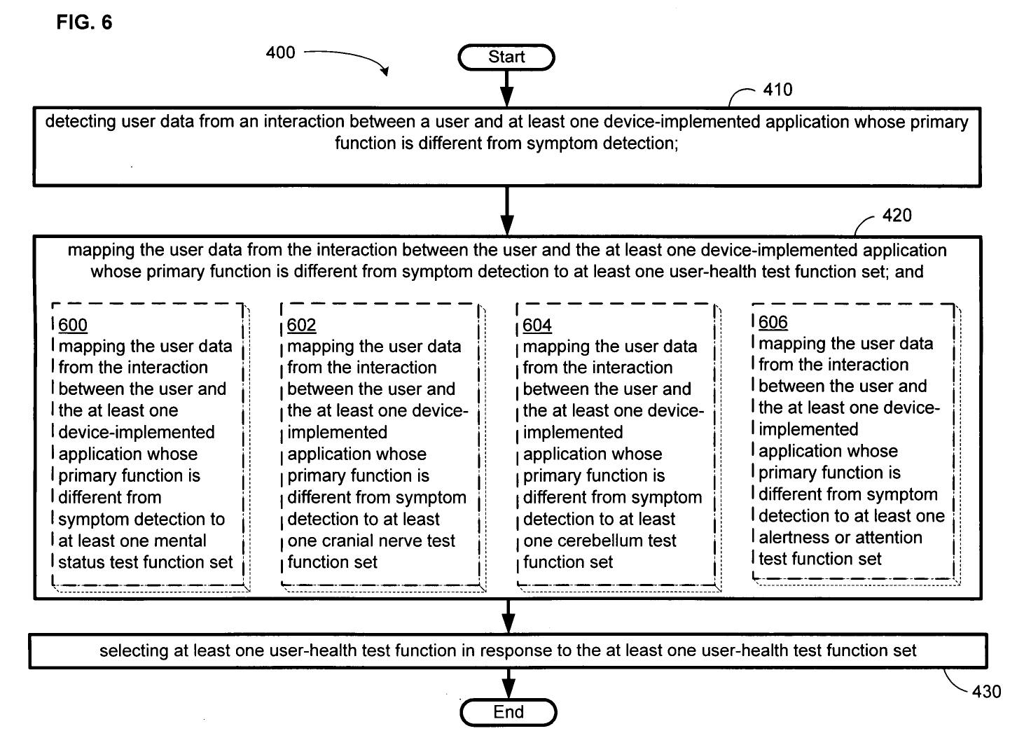

FIG. 6 illustrates alternative embodiments of the example operational flow 400 of FIG. 4. FIG. 6 illustrates example embodiments where the implementing operation 420 may include at least one additional operation. Additional operations may include operation 600, 602, 604, and/or operation 606.

Operation 600 depicts mapping the user data from the interaction between the user and the at least one device-implemented application whose primary function is different from symptom detection to at least one mental status test function set. For example, a user data mapping unit 140 may map user data 116 from the interaction between the user and the at least one device-implemented application whose primary function is different from symptom detection to a user-health test function set 197, for example including a mental status test function set within user-health test function set 197.

User data mapping to at least one mental status test function set may be done as a simple one-to-one mapping, such as for example, user reaction time data 222 mapped to a mental status analysis module 242. Alternatively, for example, user keystroke data 232 may be mapped in a one-to-many mapping, such as for example, user keystroke data 232 being mapped by user data mapping unit 140 to, for example, mental status analysis module 242, memory analysis module 254, and/or calculation analysis module 262. Alternatively, for example, user data 116 may be mapped in a many-to-one mapping. For example, user reaction time data 222, user keystroke data, and user pointing device manipulation data 234 may be mapped to an alertness or attention analysis module 248. Mapping algorithms may be applied by one of skill in the art according to known user-health test functions and those disclosed herein. For example, user speech or voice data 224 may be mapped to speech or voice analysis module 256 on the basis of the user data type itself. Alternatively, a system may be configured, for example by a user 190, to map user input data 218 to a motor skill analysis module 268 based on a user preference, such as a specific health issue like Parkinson's disease onset or risk of stroke.

A mental status test function set may include, for example, one or more alertness or attention test functions, one or more memory test functions, one more speech test functions, one or more calculation test functions, one or more neglect or construction test functions, and/or one or more sequencing task test functions.

Operation 602 depicts mapping the user data from the interaction between the user and the at least one device-implemented application whose primary function is different from symptom detection to at least one cranial nerve test function set. For example, a user data mapping unit 140 may map user data 116 from the interaction between the user and the at least one device-implemented application whose primary function is different from symptom detection to a user-health test function set 196, for example including a cranial nerve function analysis module 244.

User data mapping to at least one cranial nerve test function set may be done as a simple one-to-one mapping, such as for example, user pupil movement data mapped to a cranial nerve function analysis module 244. Alternatively, for example, user eye movement data may be mapped in a one-to-many mapping, such as for example, user eye movement data being mapped by user data mapping unit 140 to, for example, cranial nerve analysis module 244; body movement, eye movement, or pupil movement analysis module 258; and visual field analysis module 250. Alternatively, for example, user data 116 may be mapped in a many-to-one mapping. For example, user speech or voice data 224, user eye movement data, and user face movement data 230 may be mapped to a cranial nerve function analysis module 244. Mapping algorithms may be applied by one of skill in the art according to known user-health test functions and those disclosed herein. For example, user speech or voice data 224 may be mapped to speech or voice analysis module 256 on the basis of the user data type itself. Alternatively, a system may be configured, for example by a user 190, to map user speech or voice data 224 to a cranial nerve function analysis module 244 based on a user preference, such as a known health issue like a cranial nerve X (i.e., vagus nerve) lesion.

A cranial nerve test function set may include, for example, one or more visual field test functions, one or more eye movement test functions, one more pupil movement test functions, one or more face pattern test functions, one or more hearing test functions, and/or one or more voice test functions.

Operation 604 depicts mapping the user data from the interaction between the user and the at least one device-implemented application whose primary function is different from symptom detection to at least one cerebellum test function set. For example, a user data mapping unit 140 may map user data 116 from the interaction between the user and the at least one device-implemented application whose primary function is different from symptom detection to a user-health test function set 198, for example including a cerebellum function analysis module 246.

User data mapping to at least one cerebellum test function set may be done as a simple one-to-one mapping, such as for example, user pointing device manipulation data 234 mapped to a cerebellum function analysis module 246. Alternatively, for example, user data 116 may be mapped in a one-to-many mapping, such as for example, user body movement data being mapped by user data mapping unit 140 to, for example, cerebellum function analysis module 246; body movement, eye movement, or pupil movement analysis module 258; and motor skill analysis module 268. Alternatively, for example, user data 116 may be mapped in a many-to-one mapping. For example, user pointing device manipulation data 234, user body movement data, and passive user data 220 may be mapped to a cerebellum function analysis module 246. Mapping algorithms may be applied by one of skill in the art according to known user-health test functions and those disclosed herein. For example, user body movement data may be mapped to motor skill analysis module 268 on the basis of the user data type itself. Alternatively, a system may be configured, for example by a user 190, to map user pointing device manipulation data 234 to a cerebellum function analysis module 246 based on a user preference, such as a known health issue like appendicular ataxia.

A cerebellum test function set may include, for example, one or more body movement test functions and/or one or more motor skill test functions.

Operation 606 depicts mapping the user data from the interaction between the user and the at least one device-implemented application whose primary function is different from symptom detection to at least one alertness or attention test function set. For example, a user data mapping unit 140 may map user data 116 from the interaction between the user and the at least one device-implemented application whose primary function is different from symptom detection to at least one alertness or attention test function set, for example alertness or attention analysis module 248.

User data mapping to at least one alertness or attention test function set may be done as a simple one-to-one mapping, such as for example, user reaction time data 222 mapped to alertness or attention analysis module 248. Alternatively, for example, user data 116 may be mapped in a many-to-one mapping. For example, user reaction time data 222, user keystroke data, and user pointing device manipulation data 234 may be mapped to alertness or attention analysis module 248. Mapping algorithms may be applied by one of skill in the art according to known user-health test functions and those disclosed herein. For example, user speech or voice data 224 may be mapped to alertness or attention analysis module 248 on the basis of the user data type itself.

An alertness or attention test function set may include, for example, one or more reaction time test function, one or more spelling test function, and/or one more speech test function.

Alertness or attention user attributes are indicators of a user's mental status. An example of an alertness test function may be a measure of reaction time as one objective manifestation. Examples of attention test functions may include ability to focus on simple tasks, ability to spell the word “world” forward and backward, or reciting a numerical sequence forward and backward as objective manifestations of an alertness problem. An alertness or attention test module 118 and/or user-health test unit 104 may require a user to enter a password backward as an alertness test function. Alternatively, a user may be prompted to perform an executive function as a predicate to launching an application such as a word processing program. For example, an alertness test function could be activated by a user command to open a word processing program, requiring performance of, for example, a spelling task as a preliminary step in launching the word processing program. Also, writing ability may be tested by requiring the user to write their name or write a sentence on a device, perhaps with a stylus on a touchscreen.

Alternatively, a system may be configured, for example by a user 190, to map user input data 218 to an alertness or attention analysis module 248 based on a user preference, such as a specific health issue like attention deficit disorder, stroke, or dementia, as discussed below.

Reduced level of alertness or attention can indicate the following possible conditions where an acute reduction in alertness or attention is detected: stroke involving the reticular activating system, stroke involving the bilateral or unilateral thalamus, metabolic abnormalities such as hyper or hypoglycemia, toxic effects due to substance overdose (for example, benzodiazepines, or other toxins such as alcohol). Reduced level of alertness and attention can indicate the following possible conditions where a subacute or chronic reduction in alertness or attention is detected: dementia (caused by, for example, Alzheimer's disease, vascular dementia, Parkinson's disease, Huntingdon's disease, Creutzfeldt-Jakob disease, Pick disease, head injury, infection, normal pressure hydrocephalus, brain tumor, exposure to toxin (for example, lead or other heavy metals), metabolic disorders, hormone disorders, hypoxia, drug reactions, drug overuse, drug abuse, encephalitis (caused by, for example, enteroviruses, herpes viruses, or arboviruses), or mood disorders (for example, bipolar disorder, cyclothymic disorder, depression, depressive disorder NOS (not otherwise specified), dysthymic disorder, postpartum depression, or seasonal affective disorder)).

In the context of the above alertness or attention test function set, as set forth herein, available user data 116 arising from the user 190 interaction with the application 104 are one or more of various types of user data 116 described in FIG. 5 and its supporting text. A reduced level of alertness or attention may indicate certain of the possible conditions discussed above. One skilled in the art can establish or determine user-health test function sets relating to the one or more types of user data indicative of altered alertness or attention, or the one or more user-health test functions suited to evaluate altered alertness or attention that is associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

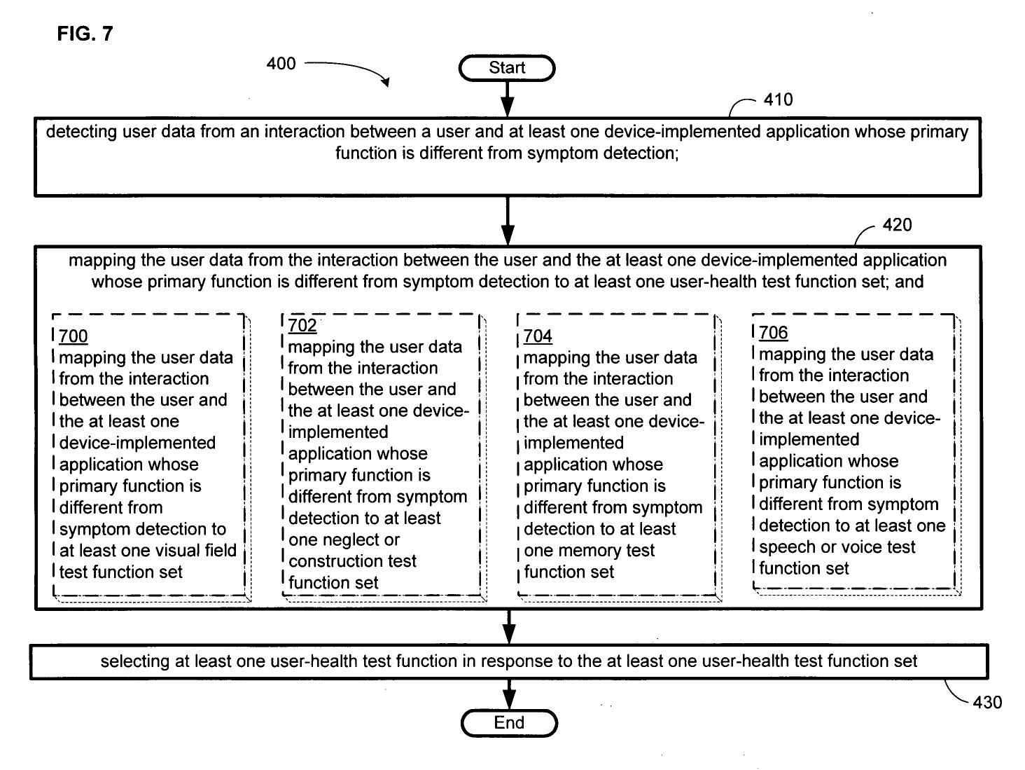

FIG. 7 illustrates alternative embodiments of the example operational flow 400 of FIG. 4. FIG. 7 illustrates example embodiments where the implementing operation 420 may include at least one additional operation. Additional operations may include operation 700, 702, 704, and/or operation 706.

Operation 700 depicts mapping the user data from the interaction between the user and the at least one device-implemented application whose primary function is different from symptom detection to at least one visual field test function set. For example, a user data mapping unit 140 may map user data 116 from the interaction between the user and the at least one device-implemented application whose primary function is different from symptom detection to at least one visual field test function set, for example visual field analysis module 250.

User data mapping to at least one visual field test function set may be done as a simple one-to-one mapping, such as for example, user pointing device manipulation data 234 mapped to visual field analysis module 250. Mapping algorithms may be applied by one of skill in the art according to known user-health test functions and those disclosed herein. Alternatively, a system may be configured, for example by a user 190, to map user input data 218 to a visual field analysis module 250 based on a user preference, such as a specific health issue like glaucoma or optic nerve lesions, as discussed below.

A visual field test function set may include, for example, one or more visual field test functions, one or more pointing device manipulation test functions, and/or one more reading test functions.

Visual field user attributes are indicators of a user's ability to see directly ahead and peripherally. An example of a visual field test function may be a measure of a user's gross visual acuity, for example using a Snellen eye chart or visual equivalent on a display. Alternatively, a campimeter may be used to conduct a visual field test. A visual field test analysis module 250 and/or user data mapping unit 140 may contain a user-health test function set 196 including a user-health test function that may prompt a user 190 to activate a portion of a display when the user 190 can detect an object entering their field of view from a peripheral location relative to a fixed point of focus, either with both eyes or with one eye covered at a time. Such testing could be done in the context of, for example, new email alerts that require clicking and that appear in various locations on a display. Based upon the location of decreased visual field, the defect can be localized, for example in a quadrant system. A pre-chiasmatic lesion results in ipsilateral eye blindness. A chiasmatic lesion can result in bi-temporal hemianopsia (i.e., tunnel vision). Post-chiasmatic lesions proximal to the geniculate ganglion can result in left or right homonymous hemianopsia. Lesions distal to the geniculate ganglion can result in upper or lower homonymous quadrantanopsia.

Visual field defects may indicate optic nerve conditions such as pre-chiasmatic lesions, which include fractures of the sphenoid bone (e.g., transecting the optic nerve), retinal tumors, or masses compressing the optic nerve. Such conditions may result in unilateral blindness and unilaterally unreactive pupil (although the pupil may react to light applied to the contralateral eye). Bi-temporal hemianopsia can be caused by glaucoma, pituitary adenoma, craniopharyngioma or saccular Berry aneurysm at the optic chiasm. Post-chiasmatic lesions are associated with homonymous hemianopsia or quadrantanopsia depending on the location of the lesion.

In the context of the above visual field test function set, as set forth herein, available user data 116 arising from the user 190 interaction with the application 104 are one or more of various types of user data 116 described in FIG. 5 and its supporting text. An altered visual field may indicate certain of the possible conditions discussed above. One skilled in the art can establish or determine user-health test function sets relating to the one or more types of user data indicative of altered visual field, or one or more user-health test functions suited to evaluate altered visual field associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 702 depicts mapping the user data from the interaction between the user and the at least one device-implemented application whose primary function is different from symptom detection to at least one neglect or construction test function set. For example, a user data mapping unit 140 may map user data 116 from the interaction between the user and the at least one device-implemented application whose primary function is different from symptom detection to at least one neglect or construction test function set, for example neglect or construction analysis module 252.

User data mapping to at least one neglect or construction test function set may be done as a simple one-to-one mapping, such as for example, user pointing device manipulation data 234 mapped to neglect or construction analysis module 252. Mapping algorithms may be applied by one of skill in the art according to known user-health test functions and those disclosed herein. Alternatively, a system may be configured, for example by a user 190, to map user input data 218 to a neglect or construction analysis module 252 based on a user preference, such as a specific health issue like stroke or brain tumor, as discussed below.

A neglect or construction test function set may include, for example, one or more body movement test functions, one or more pointing device manipulation test functions, and/or one more cognitive test functions such as drawing test functions.

Neglect or construction user attributes are indicators of a user's mental status. Neglect may include a neurological condition involving a deficit in attention to an area of space, often one side of the body or the other. A construction defect may include a deficit in a user's ability to draw complex figures or manipulate blocks or other objects in space as a result of neglect or other visuospatial impairment.

Hemineglect may include an abnormality in attention to one side of the universe that is not due to a primary sensory or motor disturbance. In sensory neglect, users ignore visual, somatosensory, or auditory stimuli on the affected side, despite intact primary sensation. This can often be demonstrated by testing for extinction on double simultaneous stimulation. Thus, a neglect or construction test function set may contain user-health test functions that present a stimulus on one or both sides of a display for a user 190 to click on. A user 190 with hemineglect may detect the stimulus on the affected side when presented alone, but when stimuli are presented simultaneously on both sides, only the stimulus on the unaffected side may be detected. In motor neglect, normal strength may be present, however, the user often does not move the affected limb unless attention is strongly directed toward it.

An example of a neglect test function may be a measure of a user's awareness of events occurring on one side of the user or the other. A user could be asked, “Do you see anything on the left side of the screen?” Users with anosognosia (i.e., unawareness of a disability) may be strikingly unaware of severe deficits on the affected side. For example, some people with acute stroke who are completely paralyzed on the left side believe there is nothing wrong and may even be perplexed about why they are in the hospital. Alternatively, a neglect or construction test function set may include a user-health test function that presents a drawing task to a user 190 in the context of an application 104 that involves similar activities. A construction test involves prompting a user to draw complex figures or to manipulate objects in space. Difficulty in completing such a test may be a result of neglect or other visuospatial impairment.

Another neglect test function is a test of a user's ability to acknowledge a series of objects on a display that span a center point on the display. For example, a user may be prompted to click on each of 5 hash marks present in a horizontal line across the midline of a display. If the user has a neglect problem, she may only detect and accordingly click on the hash marks on one side of the display, neglecting the others.

Hemineglect is most common in lesions of the right (nondominant) parietal lobe, causing users to neglect the left side. Left-sided neglect can also occasionally be seen in right frontal lesions, right thalamic or basal ganglia lesions, and, rarely, in lesions of the right midbrain. Hemineglect or difficulty with construction tasks may be indicative of stroke (e.g., embolic, thrombotic, or due to vasculitis), or brain tumor (e.g., glioma or meningioma).

In the context of the above neglect or construction test function set, as set forth herein, available user data 116 arising from the user 190 interaction with the application 104 are one or more of various types of user data 116 described in FIG. 5 and its supporting text. Altered neglect or construction attributes may indicate certain of the possible conditions discussed above. One skilled in the art can establish or determine user-health test function sets relating to the one or more types of user data indicative of altered neglect or construction function, or one or more user-health test functions suited to evaluate altered neglect or construction ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 704 depicts mapping the user data from the interaction between the user and the at least one device-implemented application whose primary function is different from symptom detection to at least one memory test function set. For example, a user data mapping unit 140 may map user data 116 from the interaction between the user and the at least one device-implemented application whose primary function is different from symptom detection to at least one memory test function set, for example memory analysis module 254.

User data mapping to at least one memory test function set may be done as a simple one-to-one mapping, such as for example, user keystroke data 232 mapped to memory analysis module 254. Alternatively, for example, user data mapping may be done as a many-to-one (or many to a few) mapping. For example, user pointing device manipulation data 234 and user keystroke data 232 may be mapped to memory analysis module 254. Mapping algorithms may be applied by one of skill in the art according to known user-health test functions and those disclosed herein. Alternatively, a system may be configured, for example by a user 190, to map user input data 218 to a memory analysis module 254 based on a user preference, such as a specific health issue like head injury or Alzheimer's disease, as discussed below.

A memory test function set may include, for example, one or more word list memory test functions, one or more number memory test functions, and/or one more personal history memory test functions. Another example of a memory test function may include a text or number input device, or user monitoring device prompting a user 190 to, for example, spell, write, speak, or calculate in order to test, for example, short-term memory, long-term memory, or the like.

A user's memory attributes are indicators of a user's mental status. An example of a memory test function may be a measure of a user's short-term ability to recall items presented, for example, in a story, or after a short period of time. Another example of a memory test function may be a measure of a user's long-term memory, for example their ability to remember basic personal information such as birthdays, place of birth, or names of relatives. A memory test function set may include a memory test function that prompts a user 190 to change and enter a password with a specified frequency during internet browser use. A memory test function involving changes to a password that is required to access an internet server can challenge a user's memory according to a fixed or variable schedule.

Difficulty with recall after about 1 to 5 minutes may indicate damage to the limbic memory structures located in the medial temporal lobes and medial diencephalon of the brain, or damage to the formix. Dysfunction of these structures characteristically causes anterograde amnesia, meaning difficulty remembering new facts and events occurring after lesion onset. Reduced short-term memory function can also indicate the following conditions: head injury, Alzheimer's disease, Herpes virus infection, seizure, emotional shock or hysteria, alcohol-related brain damage, barbiturate or heroin use, general anaesthetic effects, electroconvulsive therapy effects, stroke, transient ischemic attack (i.e., a “mini-stroke”), complication of brain surgery. Reduced long-term memory function can indicate the following conditions: Alzheimer's disease, alcohol-related brain damage, complication of brain surgery, depressive pseudodementia, adverse drug reactions (e.g., to benzodiazepines, anti-ulcer drugs, analgesics, anti-hypertensives, diabetes drugs, beta-blockers, anti-Parkinson's disease drugs, anti-emetics, anti-psychotics, or certain drug combinations, such as haloperidol and methyldopa combination therapy), multi-infarct dementia, or head injury.

In the context of the above memory test function set, as set forth herein, available user data 116 arising from the user 190 interaction with the application 104 are one or more of various types of user data 116 described in FIG. 5 and its supporting text. Altered memory attributes may indicate certain of the possible conditions discussed above. One skilled in the art can establish or determine user-health test function sets relating to the one or more types of user data indicative of altered memory function, or one or more user-health test functions suited to evaluate altered memory associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 706 depicts mapping the user data from the interaction between the user and the at least one device-implemented application whose primary function is different from symptom detection to at least one speech or voice test function set. For example, a user data mapping unit 140 may map user data 116 from the interaction between the user and the at least one device-implemented application whose primary function is different from symptom detection to at least one speech or voice test function set, for example speech or voice analysis module 256.

User data mapping to at least one speech or voice test function set may be done as a simple one-to-one mapping, such as for example, user speech or voice data 224 mapped to speech or voice analysis module 256. Mapping algorithms may be applied by one of skill in the art according to known user-health test functions and those disclosed herein. Alternatively, a system may be configured, for example by a user 190, to map user input data 218 and/or passive user data 220 to a speech or voice analysis module 256 based on a user preference, such as a specific health issue like stroke or head trauma, as discussed below.

A speech or voice test function set may include, for example, one or more speech test functions, one or more voice test functions, one more comprehension test functions, one or more naming test functions, and/or one or more reading test functions.

User speech attributes are indicators of a user's mental status. An example of a speech test function may be a measure of a user's fluency or ability to produce spontaneous speech, including phrase length, rate of speech, abundance of spontaneous speech, tonal modulation, or whether paraphasic errors (e.g., inappropriately substituted words or syllables), neologisms (e.g., nonexistent words), or errors in grammar are present. Another example of a speech test function is a program that can measure the number of words spoken by a user during a video conference. The number of words per interaction or per unit time could be measured. A marked decrease in the number of words spoken could indicate a speech problem.

Another example of a voice or speech test function may include tracking of speech or voice data into a device or user monitoring device, such as a telephonic device or a video communication device with sound receiving/transmission capability, for example when a user task requires, for example, speaking, singing, or other vocalization.

Another example of a speech test function may be a measure of a user's comprehension of spoken language, including whether a user 190 can understand simple questions and commands, or grammatical structure. For example, a user-health test function set may include a speech or voice analysis module 256 that may ask the user 190 the question “Mike was shot by John. Is John dead?” An inappropriate response may indicate a speech center defect. Alternatively a speech or voice analysis module 256 include a speech function test that may require a user to say a code or phrase and repeat it several times. Speech defects may become apparent if the user has difficulty repeating the code or phrase during, for example, a videoconference setup or while using speech recognition software.

Another example of a speech test function may be a measure of a user's ability to name simple everyday objects (e.g., pen, watch, tie) and also more difficult objects (e.g., fingernail, belt buckle, stethoscope). A speech test function may, for example, require the naming of an object prior to or during the interaction of a user 190 with an application 104, as a time-based or event-based checkpoint. For example, a user 190 may be prompted by a speech or voice test function to say “armadillo” after being shown a picture of an armadillo, prior to or during the user's interaction with, for example, a word processing or email program. A test requiring the naming of parts of objects is often more difficult for users with speech comprehension impairment. Another speech test function may, for example, gauge a user's ability to repeat single words and sentences (e.g., “no if's and's or but's”). A further example of a speech test function measures a user's ability to read single words, a brief written passage, or the front page of the newspaper aloud followed by a test for comprehension.

Difficulty with speech or reading/writing ability may indicate, for example, lesions in the dominant (usually left) frontal lobe, including Broca's area (output area); the left temporal and parietal lobes, including Wernicke's area (input area); subcortical white matter and gray matter structures, including thalamus and caudate nucleus; as well as the non-dominant hemisphere. Typical diagnostic conditions may include, for example, stroke, head trauma, dementia, multiple sclerosis, Parkinson's disease, Landau-Kleffner syndrome (a rare syndrome of acquired epileptic aphasia).

An example of a voice test function may be a measure of symmetrical elevation of the palate when the user says “aah,” or a test of the gag reflex. In an ipsilateral lesion of the vagus nerve, the uvula deviates towards the affected side. As a result of its innervation (through the recurrent laryngeal nerve) to the vocal cords, hoarseness may develop as a symptom of vagus nerve injury. A voice test module 138 and/or user-health test unit 104 may monitor user voice frequency or volume data during, for example, gaming, videoconferencing, speech recognition software use, or mobile phone use. Injury to the recurrent laryngeal nerve can occur with lesions in the neck or apical chest. The most common lesions are tumors in the neck or apical chest. Cancers may include lung cancer, esophageal cancer, or squamous cell cancer.

Other voice test functions may involve first observing the tongue (while in floor of mouth) for fasciculations. If present, fasciculations may indicate peripheral hypoglossal nerve dysfunction. Next, the user may be prompted to protrude the tongue and move it in all directions. When protruded, the tongue will deviate toward the side of a lesion (as the unaffected muscles push the tongue more than the weaker side). Gross symptoms of pathology may result in garbled sound in speech (as if there were marbles in the user's mouth). Damage to the hypoglossal nerve affecting voice/speech may indicate neoplasm, aneurysm, or other external compression, and may result in protrusion of the tongue away from side of the lesion for an upper motor neuron process and toward the side of the lesion for a lower motor neuron process. Accordingly, a voice test module 138 and/or user-health test unit 104 may assess a user's ability to make simple sounds or to say words, for example, consistently with an established voice pattern for the user.

In the context of the above speech or voice test function set, as set forth herein, available user data 116 arising from the user 190 interaction with the application 104 are one or more of various types of user data 116 described in FIG. 5 and its supporting text. Altered speech or voice attributes may indicate certain of the possible conditions discussed above. One skilled in the art can establish or determine user-health test function sets relating to the one or more types of user data indicative of altered speech or voice function, or one or more user-health test functions suited to evaluate altered speech or voice associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

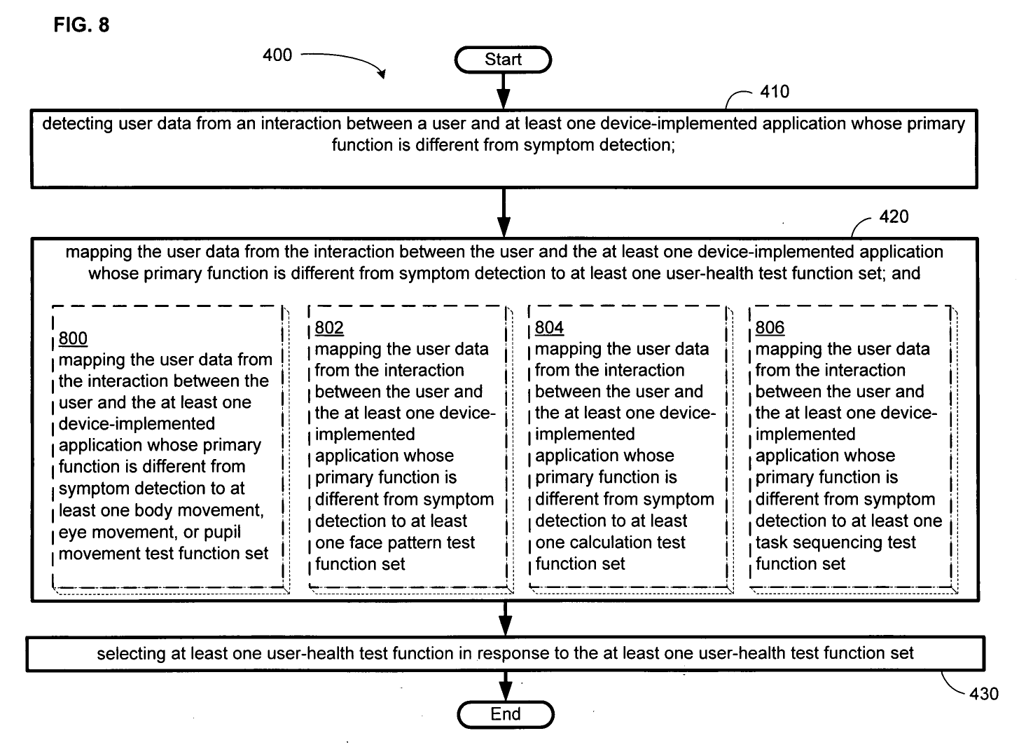

FIG. 8 illustrates alternative embodiments of the example operational flow 400 of FIG. 4. FIG. 8 illustrates example embodiments where the implementing operation 420 may include at least one additional operation. Additional operations may include operation 800, 802, 804, and/or operation 806.

Operation 800 depicts mapping the user data from the interaction between the user and the at least one device-implemented application whose primary function is different from symptom detection to at least one body movement, eye movement, or pupil movement test function set. For example, a user data mapping unit 140 may map user data 116 from the interaction between the user and the at least one device-implemented application whose primary function is different from symptom detection to at least one body movement, eye movement, or pupil movement test function set, for example body movement, eye movement, or pupil movement analysis module 258.

User data mapping to at least one body movement, eye movement, or pupil movement test function set may be done as a simple one-to-one mapping, such as for example, user body movement, eye movement, or pupil movement data 228 mapped to body movement, eye movement, or pupil movement analysis module 258. Mapping algorithms may be applied by one of skill in the art according to known user-health test functions and those disclosed herein. Alternatively, a system may be configured, for example by a user 190, to map user input data 218 and/or passive user data 220 to a body movement, eye movement, or pupil movement analysis module 256 based on a user preference, such as a specific health issue like tremor or nystagmus, as discussed below.

A body movement, eye movement, or pupil movement test function set may include, for example, one or more body movement test functions, one or more eye movement test functions, one more pupil movement test functions, and/or one or more pointing device manipulation test functions.

Another example of a body movement test function may include prompting a user 190 to activate or click a specific area on a display to test, for example, visual field range or motor skill function. Another example is visual tracking of a user's body, for example during a videoconference, wherein changes in facial movement, limb movement, or other body movements are detectable.

Another example of a body movement test function may be first observing the user for atrophy or fasciculation in the trapezius muscles, shoulder drooping, or displacement of the scapula. A body movement test function set may include a body movement test function that may then prompt the user to turn the head and shrug shoulders against resistance. Weakness in turning the head in one direction may indicate a problem in the contralateral spinal accessory nerve, while weakness in shoulder shrug may indicate an ipsilateral spinal accessory nerve lesion. Ipsilateral paralysis of the stemocleidomastoid and trapezius muscles due to neoplasm, aneurysm, or radical neck surgery also may indicate damage to the spinal accessory nerve. A body movement test function set may include a body movement test function that can perform gait analysis, for example, in the context of a security system surveillance application involving video monitoring of the user.

Cerebellar disorders can disrupt body coordination or gait while leaving other motor functions relatively intact. The term ataxia is often used to describe the abnormal movements seen in coordination disorders. In ataxia, there are medium- to large-amplitude involuntary movements with an irregular oscillatory quality superimposed on and interfering with the normal smooth trajectory of movement. Overshoot is also commonly seen as part of ataxic movements and is sometimes referred to as “past pointing” when target-oriented movements are being discussed. Another feature of coordination disorders is dysdiadochokinesia (i.e., abnormal alternating movements). Cerebellar lesions can cause different kinds of coordination problems depending on their location. One important distinction is between truncal ataxia and appendicular ataxia. Appendicular ataxia affects movements of the extremities and is usually caused by lesions of the cerebellar hemispheres and associated pathways. Truncal ataxia affects the proximal musculature, especially that involved in gait stability, and is caused by midline damage to the cerebellar vermis and associated pathways.

A body movement user-health test function set may also include a user-health test function of fine movements of the hands and feet. Rapid alternating movements, such as wiping one palm alternately with the palm and dorsum of the other hand, may be tested as well. A common test of coordination is the finger-nose-finger test, in which the user is asked to alternately touch their nose and an examiner's finger as quickly as possible. Ataxia may be revealed if the examiner's finger is held at the extreme of the user's reach, and if the examiner's finger is occasionally moved suddenly to a different location. Overshoot may be measured by having the user raise both arms suddenly from their lap to a specified level in the air. In addition, pressure can be applied to the user's outstretched arms and then suddenly released. Alternatively, testing of fine movements of the hands may be tested by measuring a user's ability to make fine movements of a cursor on a display. To test the accuracy of movements in a way that requires very little strength, a user can be prompted to repeatedly touch a line drawn on the crease of the user's thumb with the tip of their forefinger; alternatively, a user may be prompted to repeatedly touch an object on a touchscreen display.

Normal performance of motor tasks depends on the integrated functioning of multiple sensory and motor subsystems. These include position sense pathways, lower motor neurons, upper motor neurons, the basal ganglia, and the cerebellum. Thus, in order to convincingly demonstrate that abnormalities are due to a cerebellar lesion, one should first test for normal joint position sense, strength, and reflexes and confirm the absence of involuntary movements caused by basal ganglia lesions. As discussed above, appendicular ataxia is usually caused by lesions of the cerebellar hemispheres and associated pathways, while truncal ataxia is often caused by damage to the midline cerebellar vermis and associated pathways.

Another body movement test is the Romberg test, which may indicate a problem in the vestibular or proprioception system. A user is asked to stand with feet together (touching each other). Then the user is prompted to close their eyes. If a problem is present, the user may begin to sway or fall. With the eyes open, three sensory systems provide input to the cerebellum to maintain truncal stability. These are vision, proprioception, and vestibular sense. If there is a mild lesion in the vestibular or proprioception systems, the user is usually able to compensate with the eyes open. When the user closes their eyes, however, visual input is removed and instability can be brought out. If there is a more severe proprioceptive or vestibular lesion, or if there is a midline cerebellar lesion causing truncal instability, the user will be unable to maintain this position even with their eyes open.

An example of a pupil movement test function may be a measure of a user's pupils when exposed to light or objects at various distances. A pupillary movement test may assess the size and symmetry of a user's pupils before and after a stimulus, such as light or focal point. Anisocoria (i.e., unequal pupils) of up to 0.5 mm is fairly common, and is benign provided pupillary reaction to light is normal. Pupillary reflex can be tested in a darkened room by shining light in one pupil and observing any constriction of the ipsilateral pupil (direct reflex) or the contralateral pupil (contralateral reflex). If abnormality is found with light reaction, pupillary accommodation can be tested by having the user focus on an object at a distance, then focus on the object at about 10 cm from the nose. Pupils should converge and constrict at close focus.

Pupillary abnormalities may be a result of either optic nerve or oculomotor nerve lesions. An optic nerve lesion (e.g., blind eye) will not react to direct light and will not elicit a consensual pupillary constriction, but will constrict if light is shown in the opposite eye. A Horner's syndrome lesion (sympathetic chain lesion) can also present as a pupillary abnormality. In Horner's syndrome, the affected pupil is smaller but constricts to both light and near vision and may be associated with ptosis and anhydrosis. In an oculomotor nerve lesion, the affected pupil is fixed and dilated and may be associated with ptosis and lateral deviation (due to unopposed action of the abducens nerve). Small pupils that do not react to light but do constrict with near vision (i.e., accommodate but do not react to light) can be seen in central nervous system syphilis (“Argyll Robertson pupil”).

Pupillary reflex deficiencies may indicate damage to the oculomotor nerve in basilar skull fracture or uncal herniation as a result of increased intracranial pressure. Masses or tumors in the cavernous sinus, syphilis, or aneurysm may also lead to compression of the oculomotor nerve. Injury to the oculomotor nerve may result in ptosis, inferolateral displacement of the ipsilateral eye (which can present as diplopia or strabismus), or mydriasis.

An example of an eye movement test function may be a measurement of a user's ability to follow a target on a display with her eyes throughout a 360° range. Such testing may be done in the context of a user playing a game or participating in a videoconference. In such examples, user data 116 may be obtained through a camera in place as a user monitoring device 182 that can monitor the eye movements of the user during interaction with the application 104.

Another example of an eye movement test function may include eye tracking data from a user monitoring device, such as a video communication device, for example, when a user task requires tracking objects on a display, reading, or during resting states between activities in an application. A further example includes pupil movement tracking data from the user 190 at rest or during an activity required by an application or user-health test function.

Testing of the trochlear nerve or the abducens nerve for damage may involve measurement of extraocular movements. The trochlear nerve performs intorsion, depression, and abduction of the eye. A trochlear nerve lesion may present as extorsion of the ipsilateral eye and worsened diplopia when looking down. Damage to the abducens nerve may result in a decreased ability to abduct the eye.

Abnormalities in eye movement may indicate fracture of the sphenoid wing, intracranial hemorrhage, neoplasm, or aneurysm. Such insults may present as extorsion of the ipsilateral eye. Individuals with this condition complain of worsened diplopia with attempted downgaze, but improved diplopia with head tilted to the contralateral side. Injury to the abducens nerve may be caused by aneurysm, a mass in the cavernous sinus, or a fracture of the skull base. Such insults may result in extraocular palsy defined by medial deviation of the ipsilateral eye. Users with this condition may present with diplopia that improves when the contralateral eye is abducted.

Nystagmus is a rapid involuntary rhythmic eye movement, with the eyes moving quickly in one direction (quick phase), and then slowly in the other direction (slow phase). The direction of nystagmus is defined by the direction of its quick phase (e.g., right nystagmus is due to a right-moving quick phase). Nystagmus may occur in the vertical or horizontal directions, or in a semicircular movement. Terminology includes downbeat nystagmus, upbeat nystagmus, seesaw nystagmus, periodic alternating nystagmus, and pendular nystagmus. There are other similar alterations in periodic eye movements (saccadic oscillations) such as opsoclonus or ocular flutter. One can think of nystagmus as the combination of a slow adjusting eye movement (slow phase) as would be seen with the vestibulo-ocular reflex, followed by a quick saccade (quick phase) when the eye has reached the limit of its rotation.

In medicine, the clinical importance of nystagmus is that it indicates that the user's spatial sensory system perceives rotation and is rotating the eyes to adjust. Thus it depends on the coordination of activities between two major physiological systems: the vision and the vestibular apparatus (which controls posture and balance). This may be physiological (i.e., normal) or pathological.

Vestibular nystagmus may be central or peripheral. Important differentiating features between central and peripheral nystagmus include the following: peripheral nystagmus is unidirectional with the fast phase opposite the lesion; central nystagmus may be unidirectional or bidirectional; purely vertical or torsional nystagmus suggests a central location; central vestibular nystagmus is not dampened or inhibited by visual fixation; tinnitus or deafness often is present in peripheral vestibular nystagmus, but it usually is absent in central vestibular nystagmus. According to Alexander's law, the nystagmus associated with peripheral lesions becomes more pronounced with gaze toward the side of the fast-beating component; with central nystagmus, the direction of the fast component is directed toward the side of gaze (e.g., left-beating in left gaze, right-beating in right gaze, and up-beating in upgaze).

Downbeat nystagmus is defined as nystagmus with the fast phase beating in a downward direction. The nystagmus usually is of maximal intensity when the eyes are deviated temporally and slightly inferiorly. With the eyes in this position, the nystagmus is directed obliquely downward. In most users, removal of fixation (e.g., by Frenzel goggles) does not influence slow phase velocity to a considerable extent, however, the frequency of saccades may diminish.

The presence of downbeat nystagmus is highly suggestive of disorders of the cranio-cervical junction (e.g., Amold-Chiari malformation). This condition also may occur with bilateral lesions of the cerebellar flocculus and bilateral lesions of the medial longitudinal fasciculus, which carries optokinetic input from the posterior semicircular canals to the third nerve nuclei. It may also occur when the tone within pathways from the anterior semicircular canals is relatively higher than the tone within the posterior semicircular canals. Under such circumstances, the relatively unopposed neural activity from the anterior semicircular canals causes a slow upward pursuit movement of the eyes with a fast, corrective downward saccade. Additional causes include demyelination (e.g., as a result of multiple sclerosis), microvascular disease with vertebrobasilar insufficiency, brain stem encephalitis, tumors at the foramen magnum (e.g., meningioma, or cerebellar hemangioma), trauma, drugs (e.g., alcohol, lithium, or anti-seizure medications), nutritional imbalances (e.g., Wemicke encephalopathy, parenteral feeding, magnesium deficiency), or heat stroke.

Upbeat nystagmus is defined as nystagmus with the fast phase beating in an upward direction. Daroff and Troost described two distinct types. The first type consists of a large amplitude nystagmus that increases in intensity with upward gaze. This type is suggestive of a lesion of the anterior vermis of the cerebellum. The second type consists of a small amplitude nystagmus that decreases in intensity with upward gaze and increases in intensity with downward gaze. This type is suggestive of lesions of the medulla, including the perihypoglossal nuclei, the adjacent medial vestibular nucleus, and the nucleus intercalatus (structures important in gaze-holding). Upbeat nystagmus may also be an indication of benign paroxysmal positional vertigo.

Torsional (rotary) nystagmus refers to a rotary movement of the globe about its anteroposterior axis. Torsional nystagmus is accentuated on lateral gaze. Most nystagmus resulting from dysfunction of the vestibular system has a torsional component superimposed on a horizontal or vertical nystagmus. This condition occurs with lesions of the anterior and posterior semicircular canals on the same side (e.g., lateral medullary syndrome or Wallenberg syndrome). Lesions of the lateral medulla may produce a torsional nystagmus with the fast phase directed away from the side of the lesion. This type of nystagmus can be accentuated by otolithic stimulation by placing the user on their side with the intact side down (e.g., if the lesion is on the left, the nystagmus is accentuated when the user is placed on his right side).

This condition may occur when the tone within the pathways of the posterior semicircular canals is relatively higher than the tone within the anterior semicircular canals, and it can occur from lesions of the ventral tegmental tract or the brachium conjunctivum, which carry optokinetic input from the anterior semicircular canals to the third nerve nuclei.

Pendular nystagmus is a multivectorial nystagmus (i.e., horizontal, vertical, circular, and elliptical) with an equal velocity in each direction that may reflect brain stem or cerebellar dysfunction. Often, there is marked asymmetry and dissociation between the eyes. The amplitude of the nystagmus may vary in different positions of gaze. Causes of pendular nystagmus may include demyelinating disease, monocular or binocular visual deprivation, oculapalatal myoclonus, internuclear opthalmoplegia, or brain stem or cerebellar dysfunction.

Horizontal nystagmus is a well-recognized finding in patients with a unilateral disease of the cerebral hemispheres, especially with large, posterior lesions. It often is of low amplitude. Such patients show a constant velocity drift of the eyes toward the intact hemisphere with fast saccade directed toward the side of the lesion.

Seesaw nystagmus is a pendular oscillation that consists of elevation and intorsion of one eye and depression and extorsion of the fellow eye that alternates every half cycle. This striking and unusual form of nystagmus may be seen in patients with chiasmal lesions, suggesting loss of the crossed visual inputs from the decussating fibers of the optic nerve at the level of the chiasm as the cause or lesions in the rostral midbrain. This type of nystagmus is not affected by otolithic stimulation. Seesaw nystagmus may also be caused by parasellar lesions or visual loss secondary to retinitis pigmentosa.

Gaze-evoked nystagmus is produced by the attempted maintenance of an extreme eye position. It is the most common form of nystagmus. Gaze-evoked nystagmus is due to a deficient eye position signal in the neural integrator network. Thus, the eyes cannot be maintained at an eccentric orbital position and are pulled back toward primary position by the elastic forces of the orbital fascia. Then, corrective saccade moves the eyes back toward the eccentric position in the orbit.

Gaze-evoked nystagmus may be caused by structural lesions that involve the neural integrator network, which is dispersed between the vestibulocerebellum, the medulla (e.g., the region of the nucleus prepositus hypoglossi and adjacent medial vestibular nucleus “NPH/MVN”), and the interstitial nucleus of Cajal (“INC”). Patients recovering from a gaze palsy go through a period where they are able to gaze in the direction of the previous palsy, but they are unable to sustain gaze in that direction; therefore, the eyes drift slowly back toward primary position followed by a corrective saccade. When this is repeated, a gaze-evoked or gaze-paretic nystagmus results.

Gaze-evoked nystagmus often is encountered in healthy users; in which case, it is called end-point nystagmus. End-point nystagmus usually can be differentiated from gaze-evoked nystagmus caused by disease, in that the former has lower intensity and, more importantly, is not associated with other ocular motor abnormalities. Gaze-evoked nystagmus also may be caused by alcohol or drugs including anti-convulsants (e.g., phenobarbital, phenytoin, or carbamazepine) at therapeutic dosages.

Spasmus nutans is a rare condition with the clinical triad of nystagmus, head nodding, and torticollis. Onset is from age 3-15 months with disappearance by 3 or 4 years. Rarely, it may be present to age 5-6 years. The nystagmus typically consists of small-amplitude, high frequency oscillations and usually is bilateral, but it can be monocular, asymmetric, and variable in different positions of gaze. Spasmus nutans occurs in otherwise healthy children. Chiasmal, suprachiasmal, or third ventricle gliomas may cause a condition that mimics spasmus nutans.

Periodic alternating nystagmus is a conjugate, horizontal jerk nystagmus with the fast phase beating in one direction for a period of approximately 1-2 minutes. The nystagmus has an intervening neutral phase lasting 10-20 seconds; the nystagmus begins to beat in the opposite direction for 1-2 minutes; then the process repeats itself. The mechanism may be disruption of the vestibulo-ocular tracts at the pontomedullary junction. Causes of periodic alternating nystagmus may include Arnold-Chiari malformation, demyelinating disease, spinocerebellar degeneration, lesions of the vestibular nuclei, head trauma, encephalitis, syphilis, posterior fossa tumors, or binocular visual deprivation (e.g., ocular media opacities).

Abducting nystagmus of internuclear opthalmoplegia (“INO”) is nystagmus in the abducting eye contralateral to a medial longitudinal fasciculus (“MLF”) lesion.

In the context of the above body movement, eye movement, or pupil movement test function set, as set forth herein, available user data 116 arising from the user 190 interaction with the application 104 are one or more of various types of user data 116 described in FIG. 5 and its supporting text. Altered body movement, eye movement, or pupil movement attributes may indicate certain of the possible conditions discussed above. One skilled in the art can establish or determine user-health test function sets relating to the one or more types of user data indicative of altered body movement, eye movement, or pupil movement function, or one or more user-health test functions suited to evaluate altered body movement, eye movement, or pupil movement associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 802 depicts mapping the user data from the interaction between the user and the at least one device-implemented application whose primary function is different from symptom detection to at least one face pattern test function set. For example, a user data mapping unit 140 may map user data 116 from the interaction between the user and the at least one device-implemented application whose primary function is different from symptom detection to at least one face pattern test function set, for example face pattern analysis module 260.

User data mapping to at least one face pattern test function set may be done as a simple one-to-one mapping, such as for example, user face movement data 230 mapped to face pattern analysis module 260. Mapping algorithms may be applied by one of skill in the art according to known user-health test functions and those disclosed herein. Alternatively, a system may be configured, for example by a user 190, to map passive user data 220 to a face pattern analysis module 260 based on a user preference, such as a specific health issue like bell's palsy, fracture, tumor, or aneurysm, as discussed below.

A face pattern test function set may include, for example, one or more face movement test functions involving a user's ability to move the muscles of the face. An example of a face pattern test function may be a comparison of a user's face while at rest, specifically looking for nasolabial fold flattening or drooping of the corner of the mouth, with the user's face while moving certain facial features. The user may be asked to raise her eyebrows, wrinkle her forehead, show her teeth, puff out her cheeks, or close her eyes tight. Such testing may done via facial pattern recognition software used in conjunction with, for example, a videoconferencing application. Any weakness or asymmetry may indicate a lesion in the facial nerve. In general, a peripheral lesion of the facial nerve may affect the upper and lower face while a central lesion may only affect the lower face.

Abnormalities in facial expression or pattern may indicate a petrous fracture. Peripheral facial nerve injury may also be due to compression, tumor, or aneurysm. Bell's Palsy is thought to be caused by idiopathic inflammation of the facial nerve within the facial canal. A peripheral facial nerve lesion involves muscles of both the upper and lower face and can involve loss of taste sensation from the anterior ⅔ of the tongue (via the chorda tympani). A central facial nerve palsy due to tumor or hemorrhage results in sparing of upper and frontal orbicularis occuli due to crossed innervation. Spared ability to raise eyebrows and wrinkle the forehead helps differentiate a peripheral palsy from a central process. This also may indicate stroke or multiple sclerosis.

In the context of the above face pattern test function set, as set forth herein, available user data 116 arising from the user 190 interaction with the application 104 are one or more of various types of user data 116 described in FIG. 5 and its supporting text. Altered face pattern may indicate certain of the possible conditions discussed above. One skilled in the art can establish or determine user-health test function sets relating to the one or more types of user data indicative of altered face pattern, or one or more user-health test functions suited to evaluate altered face patterns associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 804 depicts mapping the user data from the interaction between the user and the at least one device-implemented application whose primary function is different from symptom detection to at least one calculation test function set. For example, a user data mapping unit 140 may map user data 116 from the interaction between the user and the at least one device-implemented application whose primary function is different from symptom detection to at least one calculation test function set, for example calculation analysis module 262.

User data mapping to at least one calculation test function set may be done as a simple one-to-one mapping, such as for example, user keystroke data 232 mapped to calculation analysis module 262. Mapping algorithms may be applied by one of skill in the art according to known user-health test functions and those disclosed herein. Alternatively, a system may be configured, for example by a user 190, to map user input data 218 to a calculation analysis module 262 based on a user preference, such as a specific health issue like stroke, brain tumor, or Gerstmann syndrome, as discussed below.

A calculation test function set may include, for example, one or more arithmetic test functions involving a user's ability to perform simple math tasks. A user's calculation attributes are indicators of a user's mental status. An example of a calculation test function may be a measure of a user's ability to do simple math such as addition or subtraction, for example. A user 190 may be prompted to solve an arithmetic problem in the context of interacting with application 104, or alternatively, in the context of using the at least one device 102 in between periods of interacting with the application 104. For example, a user may be prompted to calculate the number of items and/or gold pieces collected during a segment of gameplay in the context of playing a game. In this and other contexts, user interaction with a device's operating system or other system functions may also constitute user interaction with an application 104. Difficulty in completing calculation tests may be indicative of stroke (e.g., embolic, thrombotic, or due to vasculitis), dominant parietal lesion, or brain tumor (e.g., glioma or meningioma). When a calculation ability deficiency is found with defects in user ability to distinguish right and left body parts (right-left confusion), ability to name and identify each finger (finger agnosia), and ability to write their name and a sentence (agraphia), Gerstmann syndrome, a lesion in the dominant parietal lobe of the brain, may be present.

In the context of the above calculation test function set, as set forth herein, available user data 116 arising from the user 190 interaction with the application 104 are one or more of various types of user data 116 described in FIG. 5 and its supporting text. Altered calculation ability may indicate certain of the possible conditions discussed above. One skilled in the art can establish or determine user-health test function sets relating to the one or more types of user data indicative of altered calculation function, or one or more user-health test functions suited to evaluate altered calculation ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 806 depicts mapping the user data from the interaction between the user and the at least one device-implemented application whose primary function is different from symptom detection to at least one task sequencing test function set. For example, a user data mapping unit 140 may map user data 116 from the interaction between the user and the at least one device-implemented application whose primary function is different from symptom detection to at least one task sequencing test function set, for example task sequencing analysis module 264.

User data mapping to at least one task sequencing test function set may be done as a simple one-to-one mapping, such as for example, user keystroke data 232 mapped to task sequencing analysis module 262. Alternatively, user mapping may be done as a many-to-one mapping, for example user keystroke data 232 and user pointing device manipulation data 234 mapped to task sequencing analysis module 264. Mapping algorithms may be applied by one of skill in the art according to known user-health test functions and those disclosed herein. Alternatively, a system may be configured, for example by a user 190, to map user input data 218 to a task sequencing analysis module 264 based on a user preference, such as a specific health issue like stroke, brain tumor, or dementia, as discussed below.

A task sequencing test function set may include, for example, one or more perseveration test functions such as one or more written alternating sequencing test functions, one or more motor impersistence test functions, or one more behavior control test functions.

A user's task sequencing attributes are indicators of a user's mental status. An example of a task sequencing test function may be a measure of a user's perseveration. For example, at least one device 102 may ask a user to continue drawing a silhouette pattern of alternating triangles and squares (i.e., a written alternating sequencing task) for a time period. In users with perseveration problems, the user may get stuck on one shape and keep drawing triangles. Another common finding is motor impersistence, a form of distractibility in which users only briefly sustain a motor action in response to a command such as “raise your arms” or “look to the right.” Ability to suppress inappropriate behaviors can be tested by the auditory “Go-No-Go” test, in which the user performs a task such as moving an object (e.g., moving a finger) in response to one sound, but must keep the object (e.g., the finger) still in response to two sounds. Alternatively, at least one device 102 may prompt a user to perform a multi-step function in the context of an application 104, for example. For example, a game may prompt a user 190 to enter a character's name, equip an item from an inventory, an click on a certain direction of travel, in that order. Difficulty completing this task may indicate, for example, a frontal lobe defect associated with dementia.

Decreased ability to perform sequencing tasks may be indicative of stroke (e.g., embolic, thrombotic, or due to vasculitis), brain tumor (e.g., glioma or meningioma), or dementia (caused by, for example, Alzheimer's disease, vascular dementia, Parkinson's disease, Huntingdon's disease, Creutzfeldt-Jakob disease, Pick disease, head injury, infection (e.g., meningitis, encephalitis, HIV, or syphilis), normal pressure hydrocephalus, brain tumor, exposure to toxin (for example, lead or other heavy metals), metabolic disorders, hormone disorders, hypoxia (caused by, e.g., emphysema, pneumonia, or congestive heart failure), drug reactions (e.g., anti-cholinergic side effects, drug overuse, drug abuse (e.g., cocaine or heroin).

In the context of a task sequencing test function set, as set forth herein, available user data 116 arising from the user 190 interaction with the application 104 are one or more of various types of user data 116 described in FIG. 5 and its supporting text. Altered task sequencing ability may indicate certain of the possible conditions discussed above. One skilled in the art can establish or determine user-health test function sets relating to the one or more types of user data indicative of altered task sequencing ability, or one or more user-health test functions suited to evaluate altered task sequencing ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

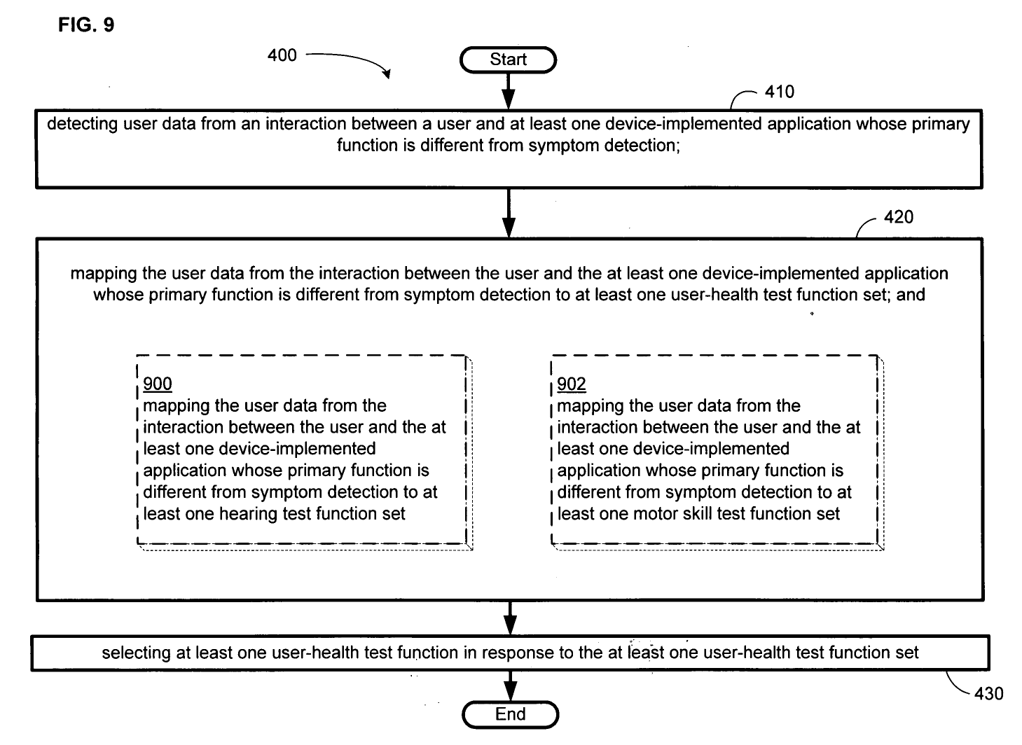

FIG. 9 illustrates alternative embodiments of the example operational flow 400 of FIG. 4. FIG. 9 illustrates example embodiments where the implementing operation 420 may include at least one additional operation. Additional operations may include operation 900 and/or operation 902.

Operation 900 depicts mapping the user data from the interaction between the user and the at least one device-implemented application whose primary function is different from symptom detection to at least one hearing test function set. For example, a user data mapping unit 140 may map user data 116 from the interaction between the user and the at least one device-implemented application whose primary function is different from symptom detection to at least one hearing test function set, for example hearing analysis module 266.

User data mapping to at least one hearing test function set may be done as a simple one-to-one mapping, such as for example, user hearing data 226 mapped to hearing analysis module 266. Alternatively, user mapping may be done as a many-to-one mapping, for example user hearing data 226 (e.g, a volume adjustment to the at least one device 102) and user input data 218 (e.g., a user action in response to a sound emanating from the at least one device 102) mapped to hearing analysis module 266. Mapping algorithms may be applied by one of skill in the art according to known user-health test functions and those disclosed herein. Alternatively, a system may be configured, for example by a user 190, to map user input data 218 and/or user hearing data 226, for example, to a hearing analysis module 266 based on a user preference, such as a specific health issue like damage to cranial nerve VIII due to skull fracture, acoustic neuroma or other tumor, ear infection, progressive deafness, or other cause of hearing loss, as discussed below.

A hearing test function set may include, for example, one or more conversation hearing test functions such as one or more tests of a user's ability to detect conversation, for example in a teleconference or videoconference scenario, one or more music detection test functions, or one more device sound effect test functions, for example in a game scenario.

An example of a hearing test function may be a gross hearing assessment of a user's ability to hear sounds. This can be done by simply presenting sounds to the user or determining if the user can hear sounds presented to each of the ears. For example, at least one device 102 may vary volume settings or sound frequency on a user's device 102 or within an application 104 over time to test user hearing. For example, a mobile phone device or other communication device may carry out various hearing test functions.

Petrous fractures that involve the vestibulocochlear nerve may result in hearing loss, vertigo, or nystagmus (frequently positional) immediately after the injury. Severe middle ear infection can cause similar symptoms but have a more gradual onset. Acoustic neuroma is associated with gradual ipsilateral hearing loss. Due to the close proximity of the vestibulocochlear nerve with the facial nerve, acoustic neuromas often present with involvement of the facial nerve. Neurofibromatosis type II is associated with bilateral acoustic neuromas. Vertigo may be associated with anything that compresses the vestibulocochlear nerve including vascular abnormalities, inflammation, or neoplasm.

In the context of a hearing test function set, as set forth herein, available user data 116 arising from the user 190 interaction with the application 104 are one or more of various types of user data 116 described in FIG. 5 and its supporting text. Altered hearing ability may indicate certain of the possible conditions discussed above. One skilled in the art can establish or determine user-health test function sets relating to the one or more types of user data indicative of altered hearing ability, or one or more user-health test functions suited to evaluate altered hearing ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 902 depicts mapping the user data from the interaction between the user and the at least one device-implemented application whose primary function is different from symptom detection to at least one motor skill test function set. For example, a user data mapping unit 140 may map user data 116 from the interaction between the user and the at least one device-implemented application whose primary function is different from symptom detection to at least one motor skill test function set, for example motor skill analysis module 268.

User data mapping to at least one motor skill test function set may be done as a simple one-to-one mapping, such as for example, user body movement data mapped to motor skill analysis module 268. Alternatively, user mapping may be done as a many-to-one mapping, for example user body movement data, user reaction time data 222, and user pointing device manipulation data 234 mapped to motor skill analysis module 268. Mapping algorithms may be applied by one of skill in the art according to known user-health test functions and those disclosed herein. Alternatively, a system may be configured, for example by a user 190, to map user input data 218 and/or passive user data 220, for example, to a motor skill analysis module 268 based on a user preference, such as a specific health issue like ataxia, tremor, or other involuntary motor defect, as discussed below.

A motor skill test function set may include, for example, one or more deliberate body movement test functions such as one or more tests of a user's ability to move an object, including objects on a display, e.g., a cursor.

An example of a motor skill test function may be a measure of a user's ability to perform a physical task. A motor skill test function may measure, for example, a user's ability to traverse a path on a display in straight line with a pointing device, to type a certain sequence of characters without error, or to type a certain number of characters without repetition. For example, a wobbling cursor on a display may indicate ataxia in the user, or a wobbling cursor while the user is asked to maintain the cursor on a fixed point on a display may indicate early Parkinson's disease symptoms. Alternatively, a user may be prompted to switch tasks, for example, to alternately type some characters using a keyboard and click on some target with a mouse. If a user has a motor skill deficiency, she may have difficulty stopping one task and starting the other task.

In clinical practice, characterization of tremor is important for etiologic consideration and treatment. Common types of tremor include resting tremor, postural tremor, action or kinetic tremor, task-specific tremor, or intention or terminal tremor. Resting tremor occurs when a body part is at complete rest against gravity. Tremor amplitude tends to decrease with voluntary activity. Causes of resting tremor may include Parkinson's disease, Parkinson-plus syndromes (e.g., multiple system atrophy, progressive supranuclear palsy, or corticobasal degeneration), Wilson's disease, drug-induced Parkinsonism (e.g., neuroleptics, Reglan, or phenthiazines), or long-standing essential tremor.

Postural tremor occurs during maintenance of a position against gravity and increases with action. Action or kinetic tremor occurs during voluntary movement. Examples of postural and action tremors may include essential tremor (primarily postural), metabolic disorders (e.g., thyrotoxicosis, pheochromocytoma, or hypoglycemia), drug-induced parkinsonism (e.g., lithium, amiodarone, or beta-adrenergic agonists), toxins (e.g., alcohol withdrawal, heavy metals), neuropathic tremor (e.g., neuropathy).

Task-specific tremor emerges during specific activity. An example of this type is primary writing tremor. Intention or terminal tremor manifests as a marked increase in tremor amplitude during a terminal portion of targeted movement. Examples of intention tremor include cerebellar tremor and multiple sclerosis tremor.

In the context of a motor skill test function set, as set forth herein, available user data 116 arising from the user 190 interaction with the application 104 are one or more of various types of user data 116 described in FIG. 5 and its supporting text. Altered motor skill ability may indicate certain of the possible conditions discussed above. One skilled in the art can establish or determine user-health test function sets relating to the one or more types of user data indicative of altered motor skill ability, or one or more user-health test functions suited to evaluate altered motor skill ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

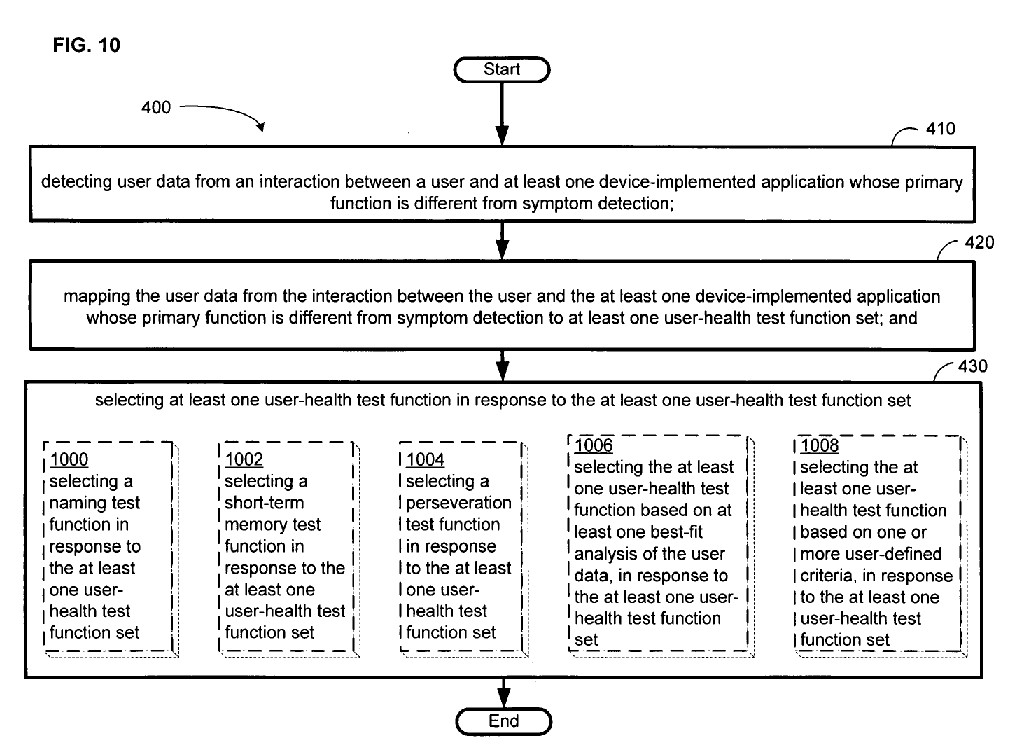

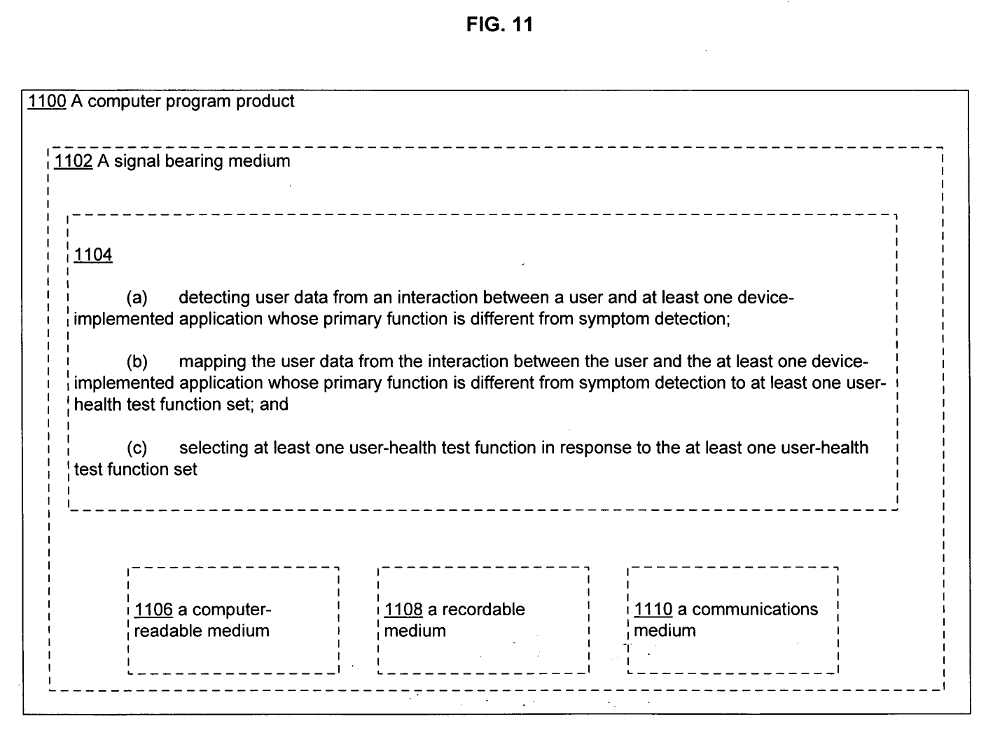

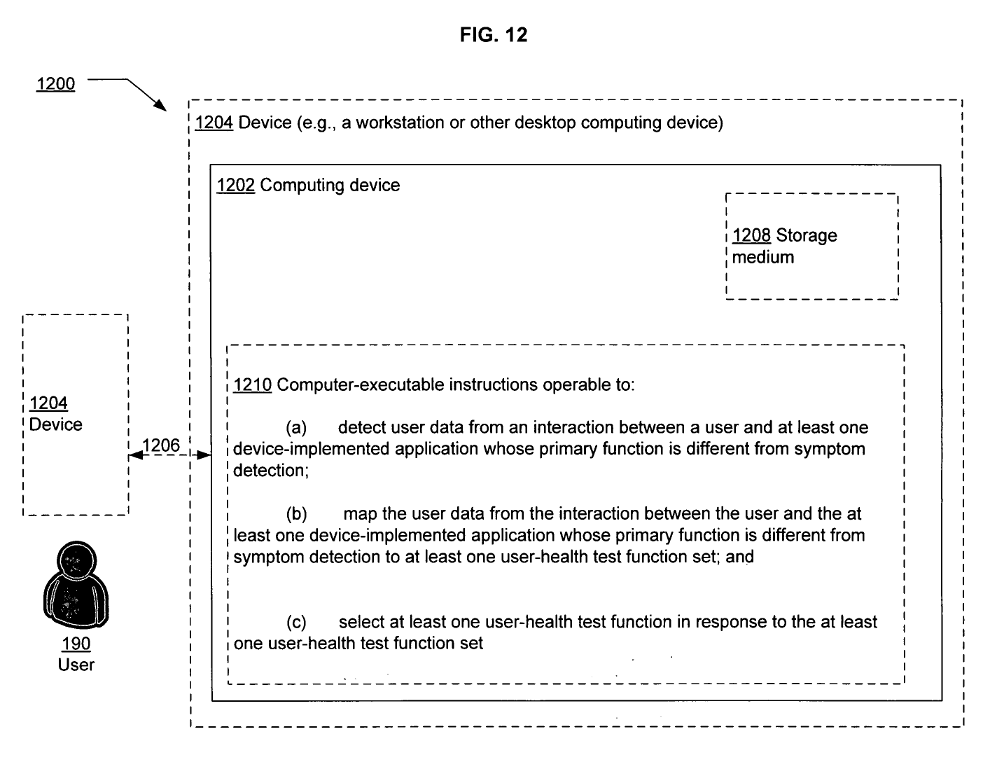

FIG. 10 illustrates alternative embodiments of the example operational flow 400 of FIG. 4. FIG. 10 illustrates example embodiments where the implementing operation 430 may include at least one additional operation. Additional operations may include operation 1000, 1002, 1004, 1006, and/or operation 1008.

Operation 1000 depicts selecting a naming test function in response to the at least one user-health test function set. For example, at least one device 102 may have installed on it at least one application 104 whose primary function is different from symptom detection, the application 104 being operable on the at least one device 102. Such an application 104 may generate user data 116 via a user input device 180, a user monitoring device 182, or a user interface 184. The at least one device 102 and/or user-health test function selection module 138 can select at least one naming test function from, for example, a user-health test function set 198 within the user data mapping unit 140.

As discussed above, a naming test function can test a user's speech ability. The at least one device 102 and/or user-health test function selection module 138 may select a naming test function in response to user data 116 being mapped to, for example a speech or voice analysis module 256.

Operation 1002 depicts selecting a short-term memory test function in response to the at least one user-health test function set. For example, at least one application 104 whose primary function is different from symptom detection may be operable on at least one device 102 through a network 192. Such an application 104 may generate user data 116 via a user input device 180, a user monitoring device 182, or a user interface 184. The at least one device 102 and/or user-health test function selection module 138 can select at least one short-term memory test function from, for example, a user-health test function set 197 within the user data mapping unit 140.

As discussed above, a short-term memory test function can test a user's memory ability. The at least one device 102 and/or user-health test function selection module 138 may select a short-term memory test function in response to user data 116 being mapped to, for example a memory analysis module 254.

Operation 1004 depicts selecting a perseveration test function in response to the at least one user-health test function set. For example, at least one application 104 whose primary function is different from symptom detection may be operable on at least one device 102 through a network 192. The at least one application 104 may be resident, for example on a server that is remote relative to the at least one device 102. Such an application 104 may generate user data 116 via a user input device 180, a user monitoring device 182, or a user interface 184. The at least one device 102 and/or a user-health test function selection module 138 can select at least one perseveration test function from, for example, a user-health test function set 196 within the user data mapping unit 140.

As discussed above, a perseveration test function can test a user's ability to perform sequencing tasks. The at least one device 102 and/or user-health test function selection module 138 may select a perseveration test function in response to user data 116 being mapped to, for example a task sequencing analysis module 264.

Operation 1006 depicts selecting the at least one user-health test function based on at least one best-fit analysis of the user data, in response to the at least one user-health test function set. For example, at least one application 104 whose primary function is different from symptom detection may be operable on at least one device 102 through a network 192. The at least one application 104 may be resident, for example, on the at least one device 102 or on a server that is remote relative to the at least one device 102. Such an application 104 may generate user data 116 via a user input device 180, a user monitoring device 182, or a user interface 184. The at least one device 102 and/or user-health test function selection module 138 can select at least one user-health test function based on at least one best-fit analysis of the user data 116, in response to, for example, user-health test function set 196 within the user data mapping unit 140.

The at least one device 102 and/or user-health test function selection module 138 may select a user-health test function from a user-health test function set to which user data 116 has been mapped on the basis of, for example, a best-fit analysis that matches a category of user data 116 with a category of user-health test function. For example, user data 116 may include user reaction time data 222 such as the speed of a user's response to a prompting icon on a display, for example, by clicking with a mouse or other pointing device, or by some other response mode. Subsequent to mapping the user reaction time data 222 to one or more user-health test function sets, the at least one device 102 and/or a user-health test function selection module 138 may perform a best-fit analysis of the user data 116 that associates the user reaction time data 222 with one or more relevant user-health test functions. This may serve as a basis for selecting one or more user-health test functions from within one or more user-health test function sets.

For example, within a game situation, a user may be prompted to click on one or more targets within the normal gameplay parameters. User reaction time data 222 may be collected once or many times for this task. The user reaction time data 222 may be mapped to mental status analysis module 242, alertness or attention analysis module 248, and/or neglect or construction analysis module 252. A best-fit analysis of the user reaction time data 222 may match data that are characteristic of a change in attention, such as loss of focus. The at least one device 102 and/or user-health test function selection module 138 may therefore select a user-health test function to test user attention, such as a test of the user's ability to accurately click a series of targets on a display within a period of time.

Accordingly, such a best-fit analysis may be used to exclude from selection one or more user-health test functions within one or more user-health test function sets to which user data 116 has been mapped. For example, the at least one device 102 and/or user-health test function selection module 138 may perform a best-fit analysis of user keystroke data 232 mapped to, for example, a memory analysis module 254, a calculation analysis module 262, and a task sequencing analysis module. The at least one device 102 and/or a user-health test function selection module 138 may determine that the nature of the keystroke data 232 is primarily text, and, in the context of a speech recognition program performing word processing or email functions, therefore a calculation test function from the calculation analysis module 262 is not appropriate for selection, or that specific arithmetic test functions within the calculation analysis module 262 are not appropriate for selection. In this example, however, a best-fit analysis may indicate that a text-based calculation test function is appropriate for selection based on the textual nature of the user keystroke data 232 (e.g., “if there are two engineers driving a train and there are five passengers on the train, how many people are on the train?”).

In another embodiment, the at least one device 102 and/or user-health test function selection module 138 may include a specific diagnosis in a best-fit analysis function. For example, as discussed above, a constellation of four kinds of altered user data 116 may indicate Gerstmann Syndrome; namely calculation deficit, right-left confusion, finger agnosia, and agraphia. Accordingly, the at least one device 102 and/or user-health test function selection module 138 may use a best-fit analysis that can select a group of user-health test functions to investigate the user's Gerstmann Syndrome profile when user data 116 is mapped to the corresponding user-health test function sets, e.g., calculation analysis module 262 (containing, e.g., calculation deficit tests), neglect and construction analysis module 252 (containing, e.g., right-left confusion tests), and speech or voice analysis module 256 (containing, e.g., finger agnosia tests and agraphia or writing tests).

Various best-fit analysis methods are known in the art and can be employed or adapted by one of skill in the art (see, for example, Zhou G., U.S. Pat. No. 6,999,931 “Spoken dialog system using a best-fit language model and best-fit grammar”).

Operation 1008 depicts selecting the at least one user-health test function based on one or more user-defined criteria, in response to the at least one user-health test function set. For example, at least one application 104 whose primary function is different from symptom detection may be operable on at least one device 102 through a network 192. The at least one application 104 may be resident, for example on a server that is remote relative to the at least one device 102. Such an application 104 may generate user data 116 via a user input device 180, a user monitoring device 182 or a user interface 184. The at least one device 102 and/or user-health test function selection module 138 can select at least one user-health test function based on one or more user-defined criteria in response to, for example, a user-health test function set 196 within the user data mapping unit 140.

The at least one device 102 and/or user-health test function selection module 138 may, for example, include a user-defined criterion that dictates selection of a particular user-health test function when a particular kind of user data 116 is mapped to one or more user-health test function sets. For example, a user 190 may be interested in tracking reaction time when playing a game whenever user reaction time data 222 is mapped to a user-health test function set. In such a case, the at least one device 102 and/or user-health test function selection module 138 may select a reaction time test function from within, for example, the alertness or attention analysis module 248.

Another example may include specific diagnostic criteria, perhaps defined within the system by a healthcare provider 310. In such a case, the healthcare provider may also be a user 190, and the at least one device 102 may be also be used by another user 190 for purposes of user-health testing. For example, if a user 190 is known to have a progressive condition such as Parkinson's disease or Alzheimer's disease, a healthcare provider 310 may define criteria by which the at least one device 102 and/or user-health test function selection module 138 may select a specific user-health test function appropriate to the condition when a particular user input is detected. In the Parkinson's disease example, a resting tremor test function may be selected in all cases in which the at least one device 102 detects user body movement data or maps user data 116 to a motor skill analysis module 268. In the Alzheimer's disease example, the at least one device 102 and/or user-health test function selection module 138 may select a long-term memory test in response to user keystroke data 232 or user data 116 mapping to memory analysis module 254.

FIG. 11 illustrates a partial view of an example computer program product 1100 that includes a computer program 1104 for executing a computer process on a computing device. An embodiment of the example computer program product 1100 is provided using a signal bearing medium 1102, and may include one or more instructions for detecting user data from an interaction between a user and at least one device-implemented application whose primary function is different from symptom detection; one or more instructions for mapping the user data from the interaction between the user and the at least one device-implemented application whose primary function is different from symptom detection to at least one user-health test function set; and one or more instructions for selecting at least one user-health test function in response to the at least one user-health test function set. The one or more instructions may be, for example, computer executable and/or logic-implemented instructions. In one implementation, the signal-bearing medium 1102 may include a computer-readable medium 1106. In one implementation, the signal bearing medium 1102 may include a recordable medium 1108. In one implementation, the signal bearing medium 1102 may include a communications medium 1110.

FIG. 12 illustrates an example system 1200 in which embodiments may be implemented. The system 1200 includes a computing system environment. The system 1200 also illustrates the user 190 using a device 1204, which is optionally shown as being in communication with a computing device 1202 by way of an optional coupling 1206. The optional coupling 1206 may represent a local, wide-area, or peer-to-peer network, or may represent a bus that is internal to a computing device (e.g., in example embodiments in which the computing device 1202 is contained in whole or in part within the device 1204). A storage medium 1208 may be any computer storage media.

The computing device 1202 includes computer-executable instructions 1210 that when executed on the computing device 1202 cause the computing device 1202 to (a) detect user data from an interaction between a user and at least one device-implemented application whose primary function is different from symptom detection; (b) map the user data from the interaction between the user and the at least one device-implemented application whose primary function is different from symptom detection to at least one user-health test function set; and (c) select at least one user-health test function in response to the at least one user-health test function set. As referenced above and as shown in FIG. 12, in some examples, the computing device 1202 may optionally be contained in whole or in part within the device 1204.

In FIG. 12, then, the system 1200 includes at least one computing device (e.g., 1202 and/or 1204). The computer-executable instructions 1210 may be executed on one or more of the at least one computing device. For example, the computing device 1202 may implement the computer-executable instructions 1210 and output a result to (and/or receive data from) the computing device 1204. Since the computing device 1202 may be wholly or partially contained within the computing device 1204, the device 1204 also may be said to execute some or all of the computer-executable instructions 1210, in order to be caused to perform or implement, for example, various ones of the techniques described herein, or other techniques.

The device 1204 may include, for example, a portable computing device, workstation, or desktop computing device. In another example embodiment, the computing device 1202 is operable to communicate with the device 1204 associated with the user 190 to receive information about the input from the user 190 for performing data access and data processing and presenting an output of the user-health test function at least partly based on the user data.

Although a user 190 is shown/described herein as a single illustrated figure, those skilled in the art will appreciate that a user 190 may be representative of a human user, a robotic user (e.g., computational entity), and/or substantially any combination thereof (e.g., a user may be assisted by one or more robotic agents). In addition, a user 190, as set forth herein, although shown as a single entity may in fact be composed of two or more entities. Those skilled in the art will appreciate that, in general, the same may be said of “sender” and/or other entity-oriented terms as such terms are used herein.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

All of the above U.S. patents, U.S. patent application publications, U.S. patent applications, foreign patents, foreign patent applications and non-patent publications referred to in this specification and/or listed in any Application Data Sheet are incorporated herein by reference, to the extent not inconsistent herewith.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the art will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise. Also, use of the phrase “based on” herein includes instances where something is “at least partly based on” something else.

What is claimed is:

1-73. (canceled)

74. A system comprising:
a device configured to detect user data from an interaction between a user and at least one device-implemented application whose primary function is different from symptom detection;
a user data mapping unit; and
a user-health test function selection module.

75. The system of claim 74 wherein the device configured to detect user data from an interaction between a user and at least one device-implemented application whose primary function is different from symptom detection comprises:
a data detection module.

76. The system of claim 74 wherein the device configured to detect user data from an interaction between a user and at least one device-implemented application whose primary function is different from symptom detection comprises:
a data capture module.

77. The system of claim 74 wherein the device configured to detect user data from an interaction between a user and at least one device-implemented application whose primary function is different from symptom detection comprises:
a user input device.

78. The system of claim 74 wherein the device configured to detect user data from an interaction between a user and at least one device-implemented application whose primary function is different from symptom detection comprises:
a user monitoring device.

79. The system of claim 74 wherein the device configured to detect user data from an interaction between a user and at least one device-implemented application whose primary function is different from symptom detection comprises:
a user interface.

80. The system of claim 74 wherein the user data mapping unit comprises:
at least one user-health test function set.

81. The system of claim 74 wherein the at least one user-health test function set comprises:
a mental status analysis module.

82. The system of claim 74 wherein the at least one user-health test function set comprises:
a cranial nerve function analysis module.

83. The system of claim 74 wherein the at least one user-health test function set comprises:
a cerebellum function analysis module.

84. The system of claim 74 wherein the at least one user-health test function set comprises:
an alertness or attention analysis module.

85. The system of claim 74 wherein the at least one user-health test function set comprises:
a visual field analysis module.

86. The system of claim 74 wherein the at least one user-health test function set comprises:
a neglect or construction analysis module.

87. The system of claim 74 wherein the at least one user-health test function set comprises:
a memory analysis module.

88. The system of claim 74 wherein the at least one user-health test function set comprises:
a speech or voice analysis module.

89. The system of claim 74 wherein the at least one user-health test function set comprises:
a body movement, eye movement, or pupil movement analysis module.

90. The system of claim 74 wherein the at least one user-health test function set comprises:
a face pattern analysis module.

91. The system of claim 74 wherein the at least one user-health test function set comprises:
a calculation analysis module.

92. The system of claim 74 wherein the at least one user-health test function set comprises:
a task sequencing analysis module.

93. The system of claim 74 wherein the at least one user-health test function set comprises:
a hearing analysis module.

94. The system of claim 74 wherein the at least one user-health test function set comprises:
a motor skill analysis module.
